(19) United States
(12) Patent Application Publication
(10) Pub. No.: US 2023/0268070 A1
(43) Pub. Date: Aug. 24, 2023

(54) SYSTEMS AND METHODS FOR AT-HOME TESTING, TREATMENT, AND MONITORING

(71) Applicant: EMED LABS, LLC, Miami, FL (US)

(72) Inventors: Zachary Carl Nienstedt, Wilton Manors, FL (US); Sam Miller, Hollywood, FL (US); Michael W. Ferro, JR., Palm Beach, FL (US); Samantha Eve Rassner, Pompano Beach, FL (US); John Andrew Sands, Weston, FL (US)

(21) Appl. No.: 18/172,160

(22) Filed: Feb. 21, 2023

Related U.S. Application Data
(60) Provisional application No. 63/268,411, filed on Feb. 23, 2022.
(60) Provisional application No. 63/362,989, filed on Apr. 14, 2022.
(60) Provisional application No. 63/331,108, filed on Apr. 14, 2022.
(60) Provisional application No. 63/363,881, filed on Apr. 29, 2022.
(60) Provisional application No. 63/363,922, filed on Apr. 29, 2022.
(60) Provisional application No. 63/364,303, filed on May 6, 2022.
(60) Provisional application No. 63/351,139, filed on Jun. 10, 2022.

(51) Int. Cl. G16H 40/63; G16H 50/30

(52) U.S. Cl. CPC G16H 40/63; G16H 50/30

(57) ABSTRACT

According to some aspects, a method for at-home diagnostic testing and treatment can include receiving, from a first data source, forecast information, the forecast information indicating a projected infection trend; receiving, from a second data source, a risk profile; receiving, from a third data source, a selection of one or more individuals; converting the selection of one or more individuals to a format compatible with a fourth data source; requesting, from the fourth data source, health information associated with at least one of the one or more individuals; receiving, from the fourth data source, health information of at least one of the one or more individuals; determining, for each of the one or more individuals, a risk score, wherein the risk score is based at least in part on the health information and the forecast information; and determining that a risk score of an individual meets the risk profile.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit of U.S. Provisional Patent Application No. 63/268,411, entitled “METHOD OF REMOTE TESTING, DIAGNOSTIC AND TREATMENT”, filed Feb. 23, 2022, U.S. Provisional Patent Application No. 63/362,989, entitled “ONGOING PHYSIOLOGICAL MONITORING AND TREATMENT”, filed Apr. 14, 2022, U.S. Provisional Patent Application No. 63/363,881, entitled “ONGOING PHYSIOLOGICAL MONITORING AND TREATMENT”, filed Apr. 29, 2022, U.S. Provisional Patent Application No. 63/364,303, entitled “ONGOING PHYSIOLOGICAL MONITORING AND TREATMENT”, filed May 6, 2022, U.S. Provisional Patent Application No. 63/331,108, entitled “SYSTEMS AND METHODS FOR FACILITATING POST-TESTING USER TREATMENT AND CARE”, filed Apr. 14, 2022, U.S. Provisional Patent Application No. 63/363,922, entitled “SYSTEMS AND METHODS FOR PURCHASING AND FULFILLMENT OF PRESCRIBED PRODUCTS,” filed Apr. 29, 2022, and U.S. Provisional Patent Application No. 63/351,139, entitled “DYNAMIC OVERLAY FOR COLLABORATIVE WEB APPLICATIONS,” filed Jun. 10, 2022, the contents of each of which are incorporated by reference herein in their entirety.

### BACKGROUND

### Field

Some embodiments are directed to systems and methods for conducting remote health testing, diagnostics, and treatments. Some embodiments are directed to remote medical testing and testing platforms. Some embodiments are directed to systems, methods, and devices for providing a follow-up service to a user after the user undergoes a test, such as a medical diagnostic test. Some embodiments relate to the monitoring and adjustment of physiological indicators.

### Description of the Related Art

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance user and health provider contact, care, advice, reminders, education, intervention, monitoring, and remote admissions. Telehealth can be especially beneficial during public health emergencies such as the COVID-19 pandemic, when medical resources may be strained. During such periods, an individual may be more reluctant to travel to their healthcare provider or their healthcare provider may have no or limited availability to treat the individual.

Often, telehealth can involve the use of a user or user's personal user device, such as a smartphone, tablet laptop, personal computer, or other device. For example, a user (e.g., a patient) can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site can be avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, connecting testing users with medical providers who are needed to provide guidance and/or oversight of the testing procedures remotely, and providing follow-up care and/or monitoring.

### SUMMARY

The systems, methods, and devices described herein each have several aspects, no single one of which is solely responsible for its desirable attributes. Without limiting the scope of this disclosure, several non-limiting features will now be described briefly.

In some aspects, the techniques described herein relate to a computer system for at-home diagnostic testing and treatment, the computing system including: one or more computer data stores configured to store a plurality of computer executable instructions; and one or more hardware computer processors in communication with the one or more computer data stores and configured to execute the plurality of computer executable instructions in order to cause the computer system to: receive, from a first data source, forecast information, wherein the forecast information indicates a projected infection trend; receive, from a second data source, a risk profile; receive, from a third data source, a selection of one or more individuals; convert the selection of one or more individuals to a format compatible with a fourth data source; request, from the fourth data source, health information associated with at least one of the one or more individuals; receive, from the fourth data source, health information associated with at least one of the one or more individuals; determine, for each of the one or more individuals, a risk score, wherein the risk score is based at least in part on the health information and the forecast information; and determine that the risk score of an individual of the one or more individuals meets the risk profile.

In some aspects, the techniques described herein relate to a computer system, wherein the computer executable instructions are further configured to cause the computer system to: electronically communicate a request to send a test box to the individual.

In some aspects, the techniques described herein relate to a computer system, wherein receiving the selection of one or more individuals includes: providing a map display, wherein the map display is configured to show a visual representation of the forecast information; receiving a selection of a geographic area; and determining one or more individuals in the geographic area.

In some aspects, the techniques described herein relate to a computer system, wherein receiving health information for one or more individuals includes: determining that a query return multiple results for an individual of the one or more individuals; and determining which of the multiple results matches the individual.

In some aspects, the techniques described herein relate to a computer system, wherein determining which of the multiple results matches the individual includes: comparing information not used to request the health information.

In some aspects, the techniques described herein relate to a computer system, wherein determining which of the multiple results matches the individual includes: determining a confidence level for each match of the one or more matches; and selecting the match with a highest confidence level.

In some aspects, the techniques described herein relate to a computer system, wherein receiving health information for one or more individuals includes: determining a confidence level for health information associated with each of the one or more individuals.

In some aspects, the techniques described herein relate to a computer system, wherein receiving health information for one or more individuals includes: determining that no health information was returned for an individual of the one or more individuals; and executing a second query to request health information for the individual, wherein the second query is broader than a first query used to request health information for the one or more individuals.

In some aspects, the techniques described herein relate to a computer system, wherein the second query uses fuzzy matching.

In some aspects, the techniques described herein relate to a computer system, wherein the computer data store further includes instructions that, when executed by the one or more computer hardware processors, causing the computer system to: convert the selection of one or more individuals to a format compatible with a fifth data source; request, from the fifth data source, non-health information associated with at least one of the one or more individuals; and receive, from the fifth data source, non-health information associated with at least one of the one or more individuals, wherein the risk score is additionally determined based at least in part on the non-health information.

In some aspects, the techniques described herein relate to a computer system, wherein the non-health information includes one or more of public transit information, pharmacy location information, employment information, housing information, healthcare provider location information, or income information.

In some aspects, the techniques described herein relate to a computer system, wherein determining that the risk score of an individual of the one or more individuals meets the risk profile includes determining that the risk score is above a threshold amount.

In some aspects, the techniques described herein relate to a computer system, wherein the threshold is based at least in part on a number of test kit boxes to be shipped.

In some aspects, the techniques described herein relate to a computer system, wherein the threshold is based at least in part on an amount to be spent for test kit boxes.

In some aspects, the techniques described herein relate to a computer system, wherein the risk score indicates a likelihood that the individual will suffer a severe health consequence if the individual contracts an infectious disease.

In some aspects, the techniques described herein relate to a computer system, wherein determining the risk score includes: providing the health information and the forecast information to a machine learning model; and receiving output from the machine learning model, the output indicating a risk level for an infectious disease.

In some aspects, the techniques described herein relate to a method for at-home diagnostic testing and treatment, the computing system including: receiving, from a first data source, forecast information, wherein the forecast information indicates a projected infection trend; receiving, from a second data source, a risk profile; receiving, from a third data source, a selection of one or more individuals; converting the selection of one or more individuals to a format compatible with a fourth data source; requesting, from the fourth data source, health information associated with at least one of the one or more individuals; receiving, from the fourth data source, health information associated with at least one of the one or more individuals; determining, for each of the one or more individuals, a risk score, wherein the risk score is based at least in part on the health information and the forecast information; and determining that the risk score of an individual of the one or more individuals meets the risk profile.

In some aspects, the techniques described herein relate to a method, further including: electronically communicate a request to send a test box to the individual.

In some aspects, the techniques described herein relate to a method, wherein receiving the selection of one or more individuals includes: providing a map display, wherein the map display is configured to show a visual representation of the forecast information; receiving a selection of a geographic area; and determining one or more individuals in the geographic area.

In some aspects, the techniques described herein relate to a method, wherein receiving health information for one or more individuals includes: determining that a query return multiple results for an individual of the one or more individuals; and determining which of the multiple results matches the individual.

In some aspects, the techniques described herein relate to a method, wherein determining which of the multiple results matches the individual includes: comparing information not used to request the health information.

In some aspects, the techniques described herein relate to a method, wherein determining which of the multiple results matches the individual includes: determining a confidence level for each match of the one or more matches; and selecting the match with a highest confidence level.

In some aspects, the techniques described herein relate to a method, wherein receiving health information for one or more individuals includes: determining a confidence level for health information associated with each of the one or more individuals.

In some aspects, the techniques described herein relate to a method, wherein receiving health information for one or more individuals includes: determining that no health information was returned for an individual of the one or more individuals; and executing a second query to request health information for the individual, wherein the second query is broader than a first query used to request health information for the one or more individuals.

In some aspects, the techniques described herein relate to a method, wherein the second query uses fuzzy matching.

In some aspects, the techniques described herein relate to a method, further including: converting the selection of one or more individuals to a format compatible with a fifth data source; requesting, from the fifth data source, non-health information associated with at least one of the one or more individuals; and receiving, from the fifth data source, non-health information associated with at least one of the one or more individuals, wherein the risk score is additionally determined based at least in part on the non-health information.

In some aspects, the techniques described herein relate to a method, wherein the non-health information includes one or more of public transit information, pharmacy location information, employment information, housing information, healthcare provider location information, or income information.

In some aspects, the techniques described herein relate to a method, wherein determining that the risk score of an individual of the one or more individuals meets the risk profile includes determining that the risk score is above a threshold amount.

In some aspects, the techniques described herein relate to a method, wherein the threshold is based at least in part on a number of test kit boxes to be shipped.

In some aspects, the techniques described herein relate to a method, wherein the threshold is based at least in part on an amount to be spent for test kit boxes.

In some aspects, the techniques described herein relate to a method, wherein the risk score indicates a likelihood that the individual will suffer a severe health consequence if the individual contracts an infectious disease.

In some aspects, the techniques described herein relate to a method, wherein determining the risk score includes: providing the health information and the forecast information to a machine learning model; and receiving output from the machine learning model, the output indicating a risk level for an infectious disease.

Various combinations of the above and below recited features, embodiments, and aspects are also disclosed and contemplated by the present disclosure.

Additional embodiments of the disclosure are described below in reference to the appended claims, which may serve as an additional summary of the disclosure.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 is a block diagram illustrating an example embodiment of some of the different actors/entities that may be involved in at-home testing, treatment, or monitoring.

FIG. 2 illustrates an example flowchart of a service provider protocol according to some embodiments described herein.

FIG. 3 illustrates an example flowchart of a user at-home test and prescription protocol according to some embodiments described herein.

FIG. 4 illustrates an example flowchart of an insurance provider protocol according to some embodiments described herein.

FIG. 5 depicts a flow chart for training an artificial intelligence or machine learning model according to some embodiments.

FIG. 6 illustrates an example of training and using an AI/ML model according to some embodiments.

FIG. 7 illustrates some examples of data sources that can be used in determining risk scores and/or risk sub-scores according to some embodiments.

FIG. 8 illustrates an example process for retrieving information corresponding to a user according to some embodiments.

FIG. 9 is a block diagram illustrating an example follow-up service protocol or method.

FIG. 10 depicts an example testing and treatment process according to some embodiments.

FIGS. 11A-11E depict an example monitoring and treatment adjustment process that may be implemented by the physiological testing and treatment system according to some embodiments.

FIGS. 12A-12C depict seasonal allergy management according to some embodiments.

FIG. 13 depicts an example process for remote testing, treatment, and monitoring according to some embodiments.

FIG. 14 is a flow chart that illustrates a process that may be run on a computing system for determining a treatment plan and monitoring user performance according to some embodiments.

FIG. 15 is a flow chart that illustrates an example process for displaying overlay content on a display according to some embodiments.

FIG. 16 is a flow chart that illustrates an example process for remote control according to some embodiments.

FIG. 17 illustrates an example of a web page overlay according to some embodiments.

FIG. 18 depicts a typical online retail scenario.

FIG. 19 depicts a system according to some embodiments in which an online retailer can offer prescription products.

FIG. 20 depicts an example web page that may be provided by a third-party e-commerce site.

FIG. 21 shows an example web page that may be presented to a user of a third-party e-commerce site according to some embodiments.

FIG. 22 is a block diagram illustrating an example embodiment of a computer system configured to run software for implementing one or more embodiments of the systems, methods, and devices disclosed herein.

FIG. 23 is a block diagram illustrating an example embodiment of a computer system configured to run software for implementing one or more embodiments of the health testing and diagnostic systems, methods, and devices disclosed herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure.

As used herein, the terms member, consumer, user, patient, and individual can refer, without limitation, to patients, users of a testing and/or treatment platform, subscribers to a telehealth service, members enrolled in an insurance plan, individuals who receive medical benefits from federal, state, and/or local government programs, individuals who receive care from nonprofit organizations, e-commerce consumers, and so forth.

### Introduction

Some embodiments herein are directed to health testing, diagnostic, and treatment platform for facilitating remote health testing and treatment via remote connection of users and medical providers. At-home medical testing and treatment provides various benefits over in-person visits to medical professionals. For example, at-home medical testing and treatment provides both safety and convenience to users and medical providers. In-person visits by individuals with infectious diseases can endanger both medical professionals, as well as anyone who encounters the individuals on their way to the in-person visit. At-home testing and treatment does not involve personal contact between the user and any other individuals who may otherwise be at risk. Furthermore, at-home testing and treatment is simply more convenient, as neither medical providers nor users need to leave the safety or comfort of their home in order to administer a test or prescribe a treatment using remote testing and treatment platforms.

Additionally, because of advancements in medical and logistics technology, especially as described herein, at-home testing and treatment can now be extremely fast. In some cases, diagnostic tests can be administered and read within seconds or minutes. Other tests may require a cure time before being read or may require delivery to a laboratory to receive results, but results can still be received within days in most cases.

Applications for at-home medical testing and treatment are abundant. For example, at-home testing can be used by users to get rapid results enabling them to secure treatments for the infectious disease while such treatments remain viable. As described herein, in some cases, the potentially serious consequences of contracting an infectious disease can be mitigated if a treatment is taken within a short period of time from the point of contraction. For example, antiviral medication can be taken after contracting the influenza virus and may result in reducing or eliminating the symptoms associated with the flu if the medication is taken before the onset of serious symptoms. Often, available treatments are only effective if taken soon after contracting the illness or after the onset of symptoms, for example within the first 24-72 hours after contraction or symptom onset. In many cases, the burden on the healthcare system associated with infectious disease and the cost of hospital visits to users, insurance providers, and governments as a result of infectious disease can be significantly reduced if treatment is taken within the effective window of time. For entities that are responsible for paying the costs of hospital trips, for example, insurance providers, governments, or individuals, there is potential for cost savings if treatment can be effectively administered to users within the effective window to reduce the number of users who must be admitted to the hospital.

At-home testing and treatment may be used by sensitive individuals such as the elderly and children. At-home testing and treatment may provide a better experience for such sensitive individuals, especially if the testing procedure is uncomfortable or invasive. At-home testing and treatment can mean that the test and treatment are done in a safe, comfortable, and familiar environment, so sensitive individuals may feel less stressed and/or worried during their test, which can allow testing to proceed more smoothly. In some instances, at-home testing and treatment can be performed in a user's home, although this need not be the case in all instances. Another consideration for at-home testing and treatment is privacy. At-home testing and treatment can be private and discreet, which is ideal for high-profile individuals or sensitive individuals who want to get tested without leaving their homes. Also, accessibility considerations favor at-home testing and treatment. At-home testing and treatment can be ideal for anyone who has transportation issues and/or mobility/accessibility considerations.

### Test Delivery

While at-home testing can offer many advantages, its utility can be limited if users do not have ready access to a test when they need it. Thus, there is a need for systems and methods for providing at-home health testing, diagnostics, and/or treatment. Accordingly, systems and methods for providing a universal platform for at-home health testing, diagnostics and treatment are provided herein. In some embodiments, various tests for infectious diseases are compiled into a diagnostic box (also referred to as “box”) and delivered to users. In some embodiments, the boxes are delivered and stored by users in advance of any infectious disease warnings, such that when case numbers are increasing in a user's area, the tests are readily available for use. In some embodiments, the boxes are different for different users. For example, one box for a user may contain a certain number of each test, such as, for example, one, two, three, four, five, and/or the like if the user lives by themselves, whereas another box may contain more of each test if the user lives with multiple people. In some embodiments, boxes may contain different tests for different users in response to user feedback regarding which treatments they are willing to take. For example, one user without a history of health complications may indicate that they would not take treatment for influenza but would take treatment for the SARS-CoV-2 (COVID-19) virus, while another user may indicate that they would take treatment for any infectious disease. In some embodiments, a user's box can include a test for an infectious disease even though the user has indicated that they would not take treatment for the infectious disease. Even though a user may refuse treatment, they may be willing to take other measures such as resting, taking medications to relieve symptoms, reducing travel, or working from home for a period of time following a positive test result. In some embodiments, the box may be designed to protect the tests from exposure to the atmosphere to prevent the tests from deteriorating. In some embodiments, the box may be waterproof.

A service provider can provide a platform and/or services for managing at-home testing and treatment. For example, the service provider can provide a platform that enables medical providers, insurance companies, and patients to arrange for the shipment of boxes, to take tests, to analyze results, and so forth, as discussed in more detail below.

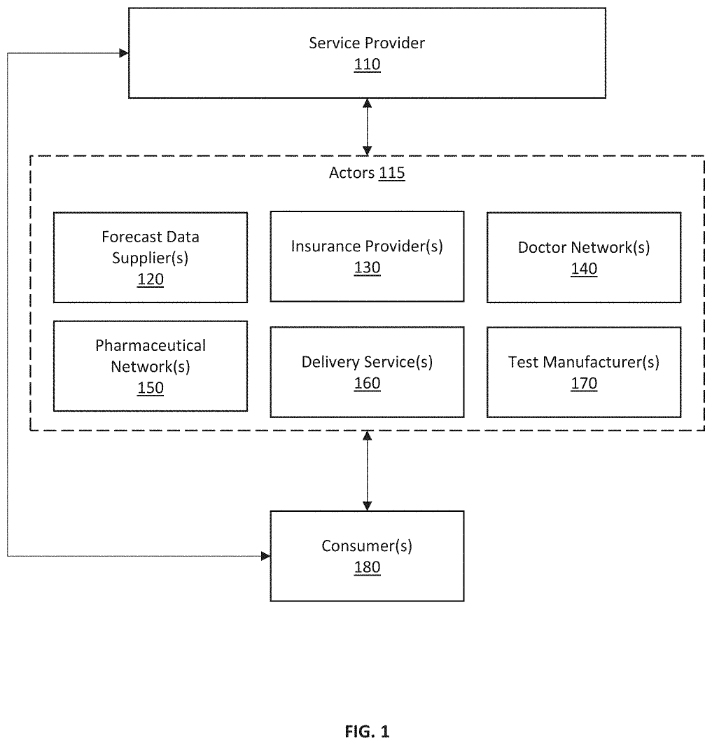

FIG. 1 is a block diagram 100 illustrating an example embodiment of some of the different actors/entities who may be working with service provider 110. Block diagram 100 includes service provider 110 and actors 115. Actors 115 can include various entities, such as forecast data supplier 120, insurance provider 130, provider network 140, pharmaceutical network 150, delivery service 160, test manufacturer 170, and consumers 180. It is understood that block diagram 100 can include additional actors not shown and, in some embodiments, not all actors shown may be required. Additionally, while block diagram 100 appears to show single actors, it is understood that each block can comprise multiple entities. For example, actors 115 can include multiple forecast data suppliers, insurance providers, doctor networks, pharmaceutical networks, delivery services, test manufacturers. There can be multiple consumers 180. Service provider 110 coordinates and works with each different actor to create a seamless process for delivering diagnostic tests and treatment to users to reduce the number of users who have serious medical complications in response to an infectious disease.

Forecast data supplier 120 may supply data and analytics to service provider 110 that may include predictions of where increased infectious disease activity will occur. For example, forecast data supplier 120 may be able to predicts trends, such as that there will be an increase in infectious disease (e.g., influenza) case numbers in a specific geographic area. A specific geographic area may be, for example, a city, county, region, state, country, and/or the like. Forecast data supplier may provide data that includes a timeframe for a predicted increase in infectious disease case numbers such as, for example, the current week, one week later, two weeks later, three weeks later, four weeks later, five weeks later, six weeks later, and/or the like. As described further herein, service provider 110 may receive data from forecast data supplier 120 and may perform additional analytics on the data. For example, service provider 110 may receive raw data from forecast supplier and may perform analytics on the raw data, such as linking infectious disease forecasts to specific geographic areas, combining the data with additional data, manipulating the data to allow it to show models or representations on a user interface, and/or the like. In some embodiments, forecast data supplier 120 may comprise one or more third parties. In some embodiments, service provider 110 may comprise forecast data supplier 120.

Insurance provider 130 may work with service provider 110 to determine which users receive boxes at which time. As described further herein, insurance provider 130 can access a forecast web application (web application) via a user interface (Forecast UI) to make informed decisions as to which members are at a high enough risk level of contracting an infectious disease or developing serious complications from a particular infectious disease to receive a box. The term “insurance provider” as used herein is a broad term and may include private insurance providers, employer insurance providers, public insurance providers (e.g., local, state, and/or federal governments), nonprofit providers, and/or the like.

Provider network 140 may comprise healthcare professionals who are qualified to give a prescription for infectious disease treatments or drugs. In some embodiments, provider network 140 may work with service provider 110 to provide prescriptions for infectious disease drugs, such as, for example, antiviral drugs for treating the influenza. In some embodiments, the provider network 140 may interact with consumers 180 over an asynchronous telehealth service. In some embodiments, the service provider platform may facilitate diagnosis of users by third-party medical providers and fulfillment of a drug prescriptions by a third-party pharmacy, without any of the parties having direct (e.g., physical or in person) contact. In some embodiments, infectious disease treatments/drugs may not require a prescription, in which case the drugs may be ordered from the pharmacy without first receiving a prescription from a health care professional.

In some embodiments described herein, prescription drug orders may be fulfilled at least in part by a third-party entity such as, for example, pharmaceutical network 150. In some embodiments, consumers 180 may be prompted to provide delivery information to the service provider platform which may be automatically transmitted to the pharmacy for fulfillment of the prescription. In some embodiments, the service provider platform may be in electronic communication with many different pharmacies and may order drugs from pharmacies by, for example, a standard electronic data transfer.

Delivery service 160 may comprise one of more delivery services that may be utilized by service provider 110 for the delivery of various products, such as, for example, boxes, drugs, and/or the like. In some embodiments, delivery service 160 may be related to the service provider platform and/or the insurance provider. In some embodiments, delivery service 160 may be a third-party delivery service scheduled by the service provider platform such as, for example, Uber™, DoorDash™, Lyft™, FedEx™, and/or the like.

Test manufacturer 170 may comprise one or more manufacturers who manufacturer one or more tests (for example, tests for infectious disease, allergies, vitamin levels, and so forth). When compiling boxes for delivery to consumers 180, service provider 110 may order various tests from test manufacturer 170 to be delivered to consumers 180. In some embodiments, test manufacturer 170 may be one or more third parties. In some embodiments, service provider 110 may manufacturer some or all of the tests themselves without use of test manufacturer 170.

While illustrated as separate blocks in FIG. 1, it will be appreciated that, as briefly mentioned above, in some embodiments, the same entity or organization can carry out the roles of multiple actors. For example, a pharmaceutical network 150 could provide delivery service 160, a test manufacturer 170 may be its own forecast data supplier 120, a provider network 140 may be closely affiliated with or part of an insurance provider 130 or a pharmaceutical network 150. Other combinations are also possible.

Consumers 180 comprise people who receive boxes and use the service provider platform to test for an infectious disease or other physiological condition, receive a diagnostic, and, if warranted, receive a treatment. As described further herein, consumers 180 may comprise members of specific insurance providers' 130 member networks. In some embodiments, consumers 180 may provide personal information to the service provider platform in order to facilitate the testing, diagnostic, and treatment of infectious disease and/or other conditions.

As shown in FIG. 1, the service provider 110 can be in communication with the actors 115 and the consumers 180. In some embodiments, actors 115 can be in communication with consumer 180 without using the service provider 110 as an intermediary. While this may be desirable in some circumstances (for example, it may be faster, may reduce demand on the service provider 110, may reduce sharing of sensitive health or other personal information, and so forth), such communication can make it more difficult for service provider 110 to keep track of events such as which consumers 180 have received boxes, which have received treatment, and so forth. In some embodiments, a process can be implemented to ensure that the service provider has information needed to maintain records.

### Service Provider Protocol

It can be important to allocate resources (e.g., tests and treatments) effectively. For example, during a surge in infectious disease cases, supplies of test kits, treatments, or both can be in high demand while supply is limited. Thus, in some embodiments, resources can be allocated to those who are most likely to benefit from them, such as people in areas with surging cases, those who are at high risk of developing severe illness or requiring hospitalization, and so forth. As discussed herein, there are many health and non-health factors that can be considered when determining how to allocate limited resources.

In some embodiments, the systems and methods herein can enable tests to be provided to users at any time. For example, test can be provided in preparation for potential future infectious disease outbreaks. In some embodiments, the systems and methods herein can be used to provide alerts to users if there are conditions that warrant using a test. For example, an insurance provider can arrange to have tests delivered to its members, and the members can store the tests at home until a service provider alerts the members (or a subset of the members, such as members in a particular city, state, or geographic area, members at higher risk, etc.) to use the received tests. Such an approach can reduce surges in demands for test supplies and can help to ensure that tests are available when needed.

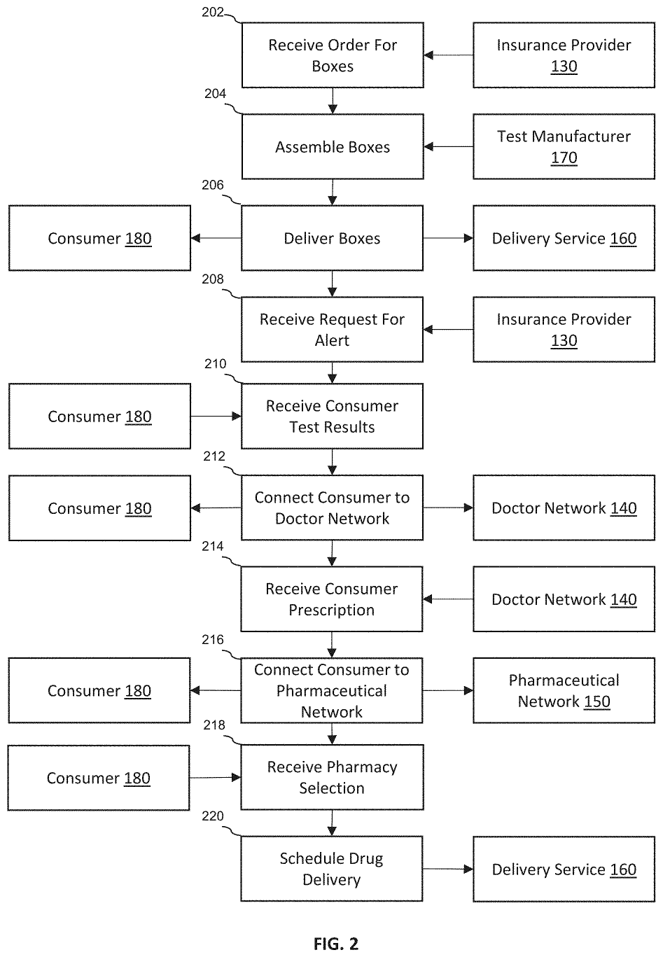

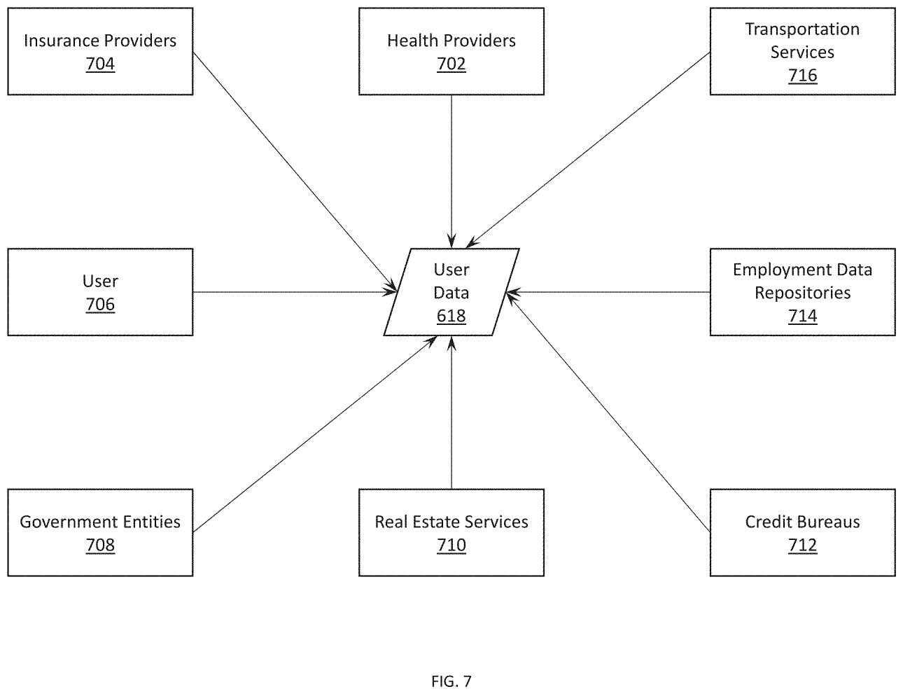

FIG. 2 illustrates an example flowchart of a service provider protocol according to some embodiments described herein. In some embodiments, the service provider protocol may be configured so that the service provider 110 is in communication with various actors 115 as described herein with reference to FIGS. 1 and 7. In some embodiments, service provider 110 may access data supplied by forecast data supplier 120. As described above, the data supplied by forecast data supplier 120 may include data predictions of increased numbers of one or more infectious diseases in particular geographic areas at particular times in the future (e.g., the next few days, the next week, the next two weeks, the next month, and so forth). In some embodiments, this data can be continually accessed, analyzed, and manipulated, such that any changes to the data are immediately received and implemented into the service provider platform. In some embodiments, the data can be checked periodically, for example every day, every two days, every week, and so forth. In some embodiments, updates to the service provider web platform may be delayed in response to changes in the forecast data. For example, in some embodiments, data supplied by forecast data supplier 120 may be accessed and updates to the web platform may be made periodically, such as twice per day, once per day, once per week, and so forth. In some embodiments, as the forecast data is updated, the Forecast UI that may be accessed by insurance provider 130 can also be updated to provide more accurate predictions for insurance provider 130 analysis.

In some embodiments, the service provider protocol may begin at block 202 when service provider 110 receives, via the Forecast UI, an order for boxes from insurance provider 130. As described further herein, the order for boxes may include user data indicating which members are to receive the boxes, user addresses, specific quantities for tests to be contained in each box for different users, specific tests to be included in each box for each user, and/or other related information. In some embodiments, the Forecast UI may not be used. For example, in some embodiments, the service provider 110 can implement an application programming interface (API) that enables ordering without use of the Forecast UI.

At block 204, the boxes may be assembled. Assembly of the boxes may include placing certain amount of infectious disease tests in each box, providing a machine-scannable image on the outside of each box (e.g., a barcode, QR code, or other pattern), and sealing the boxes for delivery. In some embodiments, at block 204, service provider 110 may order tests from one or more test manufacturers 170 prior to assembling the boxes for delivery. In some embodiments, service provider 110 may have stores of tests ready to be placed in boxes prior to delivery. In some embodiments, assembly of the boxes may be done by a third party working with the test manufacturers 170. In some embodiments, the service provider 110 may coordinate to have the test manufacturer 170 deliver tests to the third party for assembly. In some embodiments, the delivery service 160 can receive tests from test manufacturer 170, the service provider 110, or both and assemble the boxes. At block 206, the boxes can be delivered to the consumers 180 at their addresses. In some embodiments, delivery of the boxes may be done by delivery service 160.

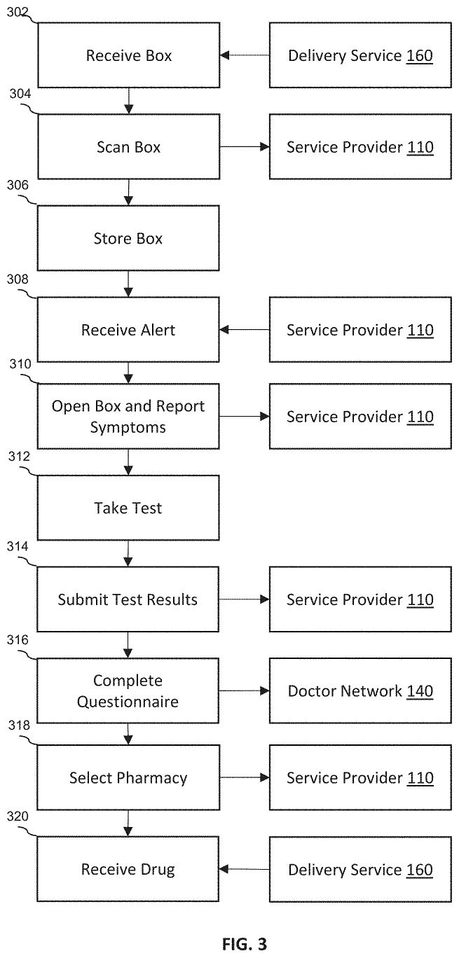

At block 208, service provider 110 receives a request to send an alert to certain users from insurance provider 130. In some embodiments, the request to send an alert may be received via a web application, such as the Forecast UI described herein. In some embodiments, the service provider 110 may implement an API that enable third parties such as insurance provider 130 to submit a request to send an alert. In response to receiving the request for an alert, service provider 110 may transmit an alert to consumer 180 via, for example, short message service (SMS), text message, electronic mail (E-mail), voice call, in-app alert, or another electronic communication channel. As described further herein, the alert may indicate that there are high numbers of an infectious disease being reported in the user's area. In some embodiments, the alert may provide consumers 180 with a list of common symptoms associated with the infectious disease. More information is provided about the alert below with reference to the user protocol and FIG. 3.

At 210, service provider 110 may receive test results from consumer 180 to the platform and process the results. As described further below, the service provider platform may provide consumers 180 with the option of indicating that their test was either positive, negative, or inconclusive. If the response received is a negative test result or an inconclusive test result, the service provider platform may provide further information to provide the users, such as, further information about the infectious disease (e.g., preventive measures), recommendations of when to retest, and/or the like. In some embodiments, the information can be customized for the user. For example, rather than instructing a user to retest after a certain time has passed, the information can provide a date on which the user should retest. In some embodiments, information can be customized based on the user's information. For example, if the user is an adult 20-year-old, information related to pediatric or elderly individuals can be excluded. In some embodiments, the further information can be selected from a set of approved information, since it can be important that information provided to consumers 180 be medically accurate and/or relevant to the individual. If the response received is a positive test result, service provider 110 proceeds to 212.

In some embodiments, a test result can be determined and/or verified by the service provider 110. For example, the consumer 180 can provide an image (which can be a still image, a video, and so forth) of a test strip, and the service provider 110 can determine and/or verify the result, for example using an image recognition algorithm.

At 212, service provider 110 has received a response from consumer 180 that the test result was positive or the service provider 110 has otherwise determined that the test result was positive. In some embodiments, the service provider platform can then determine which treatments are available to treat the particular infectious disease and whether a prescription is required. If a prescription for the drug is not required, service provider 110 may proceed to block 216. If a prescription is required, in some embodiments, consumer 180 may be connected to an asynchronous telehealth service, such as provider network 140. The provider network 140 may be a third-party network or may be part of service provider 110. As described further below, once consumer 180 is connected to the provider network 140, consumer 180 may be required to input further personal information in order to receive a prescription. In some embodiments, the provider network 140 can receive sufficient information from the service provider 110 such that the consumer 180 does not need to provide additional information to the provider network 140. In some embodiments, the location of consumer 180 may be factored into the determination of whether or not to connect to provider network 140. For example, some drugs may require a prescription if consumer 180 is located in State A, but not if consumer 180 is located in State B. Similarly, some countries may require a prescription for a particular drug, while other countries may allow for purchase without a prescription. In some locations, there may be restrictions on the prescribing of medications using telehealth services, which may preclude providing a prescription to a particular user. In some locations, there may be restrictions on who can prescribe certain treatments. For example, pharmacists may be permitted to prescribe a limited number of treatments, which can vary among locations. Nurse practitioners may have full or limited authority to prescribe depending upon the location. Thus, it can be important for the service provider 110 to ensure that, if a prescription is needed, consumer 180 is connected to a provider with a provider network 140 who is permitted to prescribe treatment to the consumer 180.

At block 214, after consumer 180 has completed their interaction with provider network 140, the provider network 140 may provide service provider 110 with a prescription for the drug. In some embodiments, this prescription may be sent to consumer 180 instead of or in addition to service provider 110. For example, in some instances, a consumer 180 may prefer to receive the prescription so they can take it to a local pharmacy to be filled. In such instances, the process depicted in FIG. 2 can end. In some embodiments, the service provider 110 may follow up with such a consumer 180 to confirm that the consumer 180 has obtained the treatment. In some embodiments, the service provider 110 may update records kept by the service provider 110 based on the consumer 180's response. For example, the service provider 110 can update information associated with a profile of the consumer 180.

If the process shown in FIG. 2 continues, at block 216, after service provider 110 receives the prescription, service provider 110 may use consumer 180's location and pharmaceutical network 150 to determine which pharmacies in the user's area are currently carrying the required drug. For example, if the infectious disease is the influenza virus, the service platform may check pharmacies that are currently carrying drugs, such as, for example, antiviral drugs that are used to treat influenza or COVID-19. Based on the determination of which pharmacies near consumer 180 are carrying the drug, service provider 110 may generate a list (e.g., a popup list, dropdown list, table, etc.) that can be displayed by the user device which allows consumer 180 to select which pharmacy they would like to receive the drug from. In some embodiments, the suggested pharmacies may be limited to a specific geographic area, such as, for example, 1 mile, 2 miles, 5 miles, 10 miles, and/or the like from the location of consumer 180. In some embodiments, service provider 110 can provide a map of pharmacies that can be displayed on the user device.

At block 218, service provider 110 receives the pharmacy selection from consumer 180. In response to the selection, service provider 110 may connect, via pharmaceutical network 150, with the particular pharmacy and place an order for the particular drug. In some embodiments, where a prescription is required, the prescription may also be supplied to the particular pharmacy. In some embodiments, service provider 110 may also provide a payment method to purchase the drug from the pharmacy. In some embodiments, service provider 110 may provide consumer 180 with the option of inputting a payment method to purchase the drug from the pharmacy.

At block 220, service provider 110 connects with delivery service 160 to schedule the pickup of the drug from the pharmacy and delivery of the drug to consumer 180. In some embodiments, the cost of the delivery may be passed on to the user. In some embodiments, the cost of the delivery may be directly billed to insurance provider 130.

### User Protocol

FIG. 3 illustrates an example flowchart of a user at-home test and prescription protocol according to some embodiments described herein. In some embodiments, the user protocol may require use of a user device. The user device may comprise, for example, a personal computer, a cellular phone, a smartphone, a laptop, a tablet computer, an e-reader device, an audio player, or another device capable of connecting to and communicating over a network, whether wired or wireless. In some embodiments, the user protocol may require consumer 180 to access a testing platform or a third-party site or application in connection with the service provider platform. In some embodiments, information can be gathered about consumer 180 that may facilitate various functionality of the service provider platform. For example, the user's identity can be verified. Verification of the user's identity can occur in various ways as described further below. In some embodiments, verification of the user's identity comprises checking the user's ID (e.g., driver's license or passport). In some embodiments, verification of the user's identity comprises checking the user's identity using a health card or test pass. In some embodiments, the user's identity is verified using biometrics.

In some embodiments, a medical test may require an order from a provider, such as a provider in provider network 140. In some embodiments, a medical test may not require an order from a healthcare provider, in which case a test could be directly sent to the user without involvement of a third party such as provider network 140. In some embodiments, a prescriber can be part of provider network 140. In some embodiments, the provider network 140 can be different from the service provider 110. In some embodiments, the third-party prescriber (e.g., provider network 140) could be an administrator or other employee of the provider of the of the health testing and diagnostic platform (e.g., service provider 110).

In some embodiments, the user at-home test and prescription protocol may begin at block 302 when a consumer 180 receives a box to their home. The box may contain one or more types of at-home tests for infectious diseases such as, for example, tests for the SARS-CoV-2 virus, tests for the influenza, and/or the like. The box may have a machine-scannable image on the outside, such as a QR code or bar code, that can be read using a user device, such as a smartphone camera. The box may provide other indicators that consumer 180 should scan the code with the user device.

At block 304, consumer 180 may scan the machine-scannable image, which may generate a pop-up banner, pop-up window, dialog box, or the like, that describes the user experience. When consumer 180 clicks or taps the pop-up banner or the like, the user device may be directed to service provider 110 platform page that may display instructions and information about the box. For example, consumer 180 may be informed about the tests contained in the box, consumer 180 may be informed about who provided the box, consumer 180 may be informed about why the box was provided, and/or the like. For example, the information may indicate that consumer 180 will be warned when an infectious disease is likely to be prevalent in the user's area and that the box was provided by, for example, the user's insurance provider. The information may also prompt consumer 180 to sign up or opt in to receiving alerts about infectious disease in their area. For example, consumer 180 may be asked to input a phone number, email, and/or the like to receive alerts. In some embodiments, consumer 180 may also be invited to download the service provider app. The information may also indicate that consumer 180 should store the box in their house so that it can be accessed when needed.

In some embodiments, augmented reality may be used to improve the user experience of receiving the box. For example, augmented reality may be used to virtually unpack the box and explain the benefits of the box to consumer 180. In some embodiments, augmented reality can be used to explain the contents of box. In some embodiments, augmented reality can be used to demonstrate how to use the contents of the box.

At block 306, consumer 180 may store the box in their house. In some embodiments, consumer 180 may be given the option of taking a picture or recording a note which indicates where the box is stored in the house. The picture or note may be uploaded to the app or submitted to the service provider platform so that when consumer 180 receives the alert, they can be reminded where the box is stored.

At block 308, consumer 180 may receive an alert that indicates that there are high numbers of an infectious disease being reported in the user's area. The alert may provide consumer 180 with a list of common symptoms associated with the infectious disease. For example, if the infectious disease of concern is the SARS-CoV-2 virus, the symptoms identified in the alert could include fever or chills, cough, shortness of breath or difficulty breathing, fatigue, muscle or body aches, headache, new loss of taste or smell, sore throat, congestion or runny nose, nausea or vomiting, diarrhea, and/or any other symptoms that are identified by medical professionals to be commonly associated with the virus. In some embodiments, the alert may direct consumer 180 to the webpage of a government agency associated with public health, such as, for example, the Centers for Disease Control and Prevention (CDC), where common symptoms and other information is provided. The alert may prompt consumer 180 to identify whether they are experiencing any of the common symptoms provided. Based on the prompt and the list of symptoms, consumer 180 may identify either that they have one or more of the symptoms or that they are symptom free. If consumer 180 is symptom free, the alert may instruct consumer 180 to take no further action and the alert may advise consumer 180 to continue to monitor for potential symptoms over a period of time, such as, for example, one to three days, a week, two weeks, a month, and/or the like. In some embodiments, the service provider platform may prompt consumer 180 to provide a response related to their symptoms. For example, after the alert is provided, consumer 180 may be asked to select (e.g., click) “yes” or “no” in response to a question of whether symptoms were identified. In some embodiments, if the answer is “yes”, consumer 180 may be asked to choose which symptoms they are experiencing from a list of common symptoms. If consumer 180 identifies that they are experiencing symptoms, consumer 180 proceeds to block 310.

At block 310, consumer 180 has identified that they are experiencing one or more of the common symptoms. Consumer 180 is then instructed to open the box. The instruction may be part of the alert, such as, for example, an instruction at the bottom of the alert that tells consumer 180 to open the box if they are experiencing any symptoms. In some embodiments, the instructions may be provided in response to consumer 180 answering the yes/no question about the symptoms. In some embodiments, when consumer 180 inputs a “yes”, the service provider platform may instruct consumer 180 to open the box and may provide a reminder of where the box is stored, such as providing consumer 180 with the note or picture that consumer 180 had previously submitted to service provider 110. The instructions may also inform consumer 180 of which test to retrieve from the box. For example, continuing with the SARS-CoV-2 virus example, consumer 180 may be instructed to retrieve the test for the SARS-CoV-2 virus. In some embodiments, the tests may be generically labeled based on the infectious disease, such as, for example, SARS-CoV-2/COVID-19 test, Flu Test, and/or the like. In some embodiments, the tests may be labeled based on the make or brand of the test.

At block 312, consumer 180 takes the identified test for the identified infectious disease. In some embodiments, service provider 110 may link or provide an asynchronous telehealth service to consumer 180 via the service provider platform or a third-party platform. Consumer 180 may receive instructions from the telehealth service related to taking the identified test. Consumer 180 may be invited to answer some questions related to their symptoms and/or other questions related to their health via asynchronous telehealth service. In some embodiments, the asynchronous telehealth service may be selected to be an in-network provider for the user.

At block 314, consumer 180 submits the results of the test to service provider 110. In some embodiments, submitting the results of the test may comprise a selection of whether the test was “negative” or “positive” via the service provider platform. If the result of the test is negative, consumer 180 will not proceed to the next step. In some embodiments, if a user's results are negative, service provider 110 may recommend that consumer 180 takes another test at a different time. For example, if the tests are likely to produce a false-negative, the service provider platform may recommend that consumer 180 immediately takes a second test. In some embodiments, the service provider platform may recommend that consumer 180 waits a certain amount of time before taking another test, such as, for example, 2 hours, 4 hours, 8 hours, 24 hours, 2 days, and/or the like. If the results of the test are positive, consumer 180 may proceed to block 316 if a prescription for the drug is required or block 318 if no prescription for the drug is required.

At block 316, consumer 180 may be connected to an asynchronous telehealth service, such as provider network 140 via the service provider platform or a third-party platform. In some embodiments, consumer 180 may be given the option of selecting an in-network telehealth service. Once connected to provider network 140, consumer 180 may be required to input some personal information to receive a prescription for the drug. In some embodiments, consumer 180 may be required to fill out series of questions, such as, for example, a questionnaire, to receive the prescription. In some embodiments, depending on state law, consumer 180 may be required to verify their identity before a prescription can be received. Verification of a user's identity can be accomplished in several ways. For example, consumer 180 can be asked to upload a copy of an official identification (e.g., a driver's license, passport, or the like) while connected to the provider network 140. In some embodiments, verification of identity may have already occurred as part of an account creation or pre-qualification step. In some embodiments, once the user's identity is verified, the user's identity can be associated with the user's account for future tests. For example, after verification, future verifications may be automated with face matching technology.

In another example, user identification can be achieved using biometrics. For example, in some embodiments, a consumer 180 accessing the service provider platform may have previously been given the option to perform a biometric initialization, in which consumer 180 went to a physical location where their fingerprint or a unique segment of DNA can be sampled and stored. Thereafter, every test taken can sample their fingerprint or DNA to verify identity. This may also be automated. In other embodiments, biometrics may be performed using biometric features of the user's device. For example, many smartphones today are capable of taking a user's fingerprint or recognizing a user's face. These features may be used to verify the user's identity in some embodiments.

At block 318, consumer 180 received a positive test result and submitted the result to the service provider platform. In response to receiving a positive test indicator, the service provider platform may generate a list of pharmacies in the user's area that are currently carrying drugs to treat the infectious disease. For example, the service provider platform may generate a popup banner of a pharmacy checker list. In some embodiments, consumer 180 may be able to limit the pharmacy search radius via the service provider platform. Based on the provided list of pharmacies that are carrying the required drug, consumer 180 may select a pharmacy of their choice to receive the drug from. In some embodiments, consumer 180 may also be required to input a payment method to purchase the drug from the pharmacy. In some embodiments, the service provider platform may have access to inventory information about the pharmacies. In some embodiments, the service provider platform may exclude pharmacies that are out of stock of the required drug or may provide an indication that a pharmacy is out of stock of the required drug. Inventory information can be especially valuable when the success of a treatment strongly depends on beginning treatment early after contraction of an illness or the onset of symptoms.

In some embodiments, when a positive test result is received, consumer 180 may be warned to quarantine or remain at their home for a certain amount of time. In some embodiments, the quarantine time may be based on a government agency's, such as the CDC's, recommendation. In some embodiments, the service provider platform will provide consumer 180 with more information about the infectious disease in response to a positive test result. For example, the service provider platform may provide consumer 180 with further indicators or instructions of what to do if their symptoms continue to get worse. For example, the service provider platform may recommend visiting the hospital if consumer 180 has trouble breathing over a period of time. In some embodiments the service provider platform will provide a link for consumer 180 to a government agency webpage that provides more information about the infectious disease.

At block 320, the drug may be delivered to consumer 180 by delivery service 160 scheduled by the service provider platform. In some embodiments, the drug may be delivered within a certain amount of time, such as, for example, 30 minutes, 1 hour, 2 hours, 4 hours, and/or the like. In some embodiments, the delivery time may be variable depending on the availability of the drug, the distance of the drug from the user, the current traffic conditions, and/or the like. In some embodiments, the cost of the drug and/or the delivery may be covered by the user's insurance provider. In another embodiment, consumer 180 may input a payment method to pay for the delivery. Once the drug has been delivered, consumer 180 may follow the instructions on the drug and use/take the drug.

### Insurance Provider Protocol

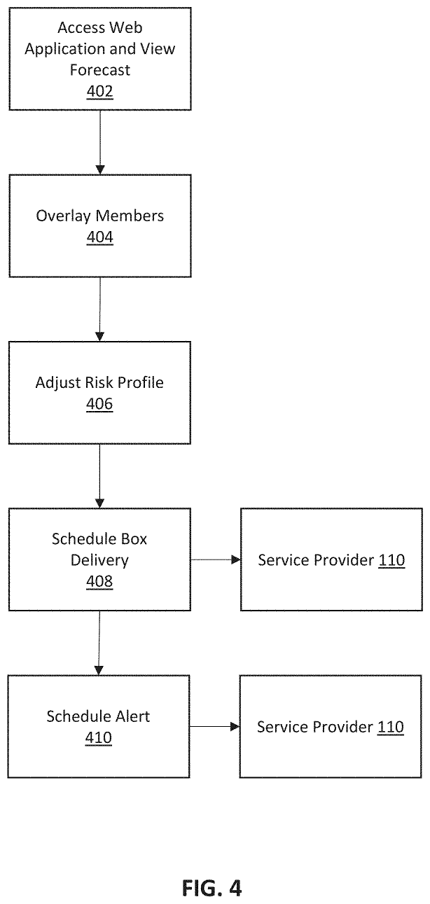

FIG. 4 illustrates an example flowchart of an insurance provider protocol according to some embodiments described herein. In some embodiments, the insurance provider protocol may require use of a provider device. The provider device may comprise, for example, a personal computer, a cellular phone, a smartphone, a laptop, a tablet computer, an e-reader device, an audio player, or another device capable of connecting to and communicating over a network, whether wired or wireless.

In some embodiments, insurance provider protocol may begin at 402 when an insurance provider, or an employee associated with the insurance provider, such as, for example, a benefits manager, accesses the service provider web application related to infectious disease forecast. As described further herein, the web application may provide a user interface (Forecast UI) that shows an infectious disease forecast for a certain geographic area. For example, the geographic area may be a city, county, region, state, country, and/or the like. In some embodiments, the insurance provider can use the Forecast UI to adjust the timeline for the forecast. For example, the Forecast UI may be adjusted to show the infectious disease forecast for, for example, the current week, one week later, two weeks later, three weeks later, four weeks later, six weeks later, and/or the like.

Some of all of the functionality of the Forecast UI as described herein can be implemented or made available in various ways. In some embodiments, the service provider 110 may provide an API so that some or all functions of the Forecast UI can be carried out without accessing the Forecast UI. This may be especially desirable for larger insurance companies or healthcare entities who have existing systems into which they wish to integrate the service provider 110's services.

At 404, the insurance provider uses the Forecast UI to overlay their members to a particular geographic area. The geographic area can include one or more towns, cities, counties, regions, statistical areas, or any other areas. In some embodiments, the area can encompass all of the insurance provider's members. Information about insurance members may be uploaded to the web application. In some embodiments, member locations may have been previously uploaded to the web application. In some embodiments, the web application may access data stored by the insurance provider in a data store to access information about the insurance provider's members. Based on the location of the members, the insurance provider can see via the Forecast UI if any members are located in an area that is likely to have high case numbers of a certain infectious disease sometime in the future. Based on this information, the insurance provider can decide whether to proceed to 406. For example, if an insurance provider only has members in State A, and the infectious disease forecast indicates low likelihood of high case numbers in State A, the insurance provider may choose not to proceed to 406. In the alternative, if the infectious disease forecast indicates a likelihood of high case numbers, the insurance provider may choose to proceed to 406.

At 406, the insurance provider may adjust the risk profile in the Forecast UI. In some embodiments, adjusting the risk profile may require accessing personal information about members such as, for example, age, sex, weight, race, previous medical conditions, and/or the like. Based on the member personal information and information about the particular infectious disease, the service provider web application may perform analytics to determine a risk score associated with each member. For example, if the infectious disease is influenza, the web application may use information related to influenza to determine what types of personal information makes a person more likely to contract influenza and/or suffer serious consequences such as, for example, permanent health conditions, hospitalization, death, vaccination status, and/or the like. For example, a user who has been vaccinated for influenza may be at less risk than a similar user who has not been vaccinated. Continuing with the influenza example, the web application may store data that suggests, for example, children under 5 years old, and adults older than 65 years old may suffer more serious health consequences as a result of contracting the influenza virus. Other stored data may suggest that members with a history of asthma, heart disease, strokes, diabetes, HIV/AIDS, cancer, neurologic conditions, chronic kidney disease, blood disorders, and/or the like are more likely to more serious health consequences as a result of contracting the influenza virus. In some embodiments, member living conditions may also be a factor considered. For example, if a member lives in nursing home or military barrack, there may be an increased risk of contracting the infectious disease. The increased risk may be factored into the risk score. Based on this data and similar data, each member may receive a risk score. For example, a 20-year-old living in a house may have a low risk score, a 50-year old with a history of heart disease may have a higher score, and a 95-year old with cancer and heart disease may have an even higher score, although the increased risk can, in some cases, be mitigated because the individual may rely on grocery delivery, may not leave the home often, and so forth, which can reduce the risk of exposure to infectious disease. Because it may not always be economically feasible or helpful to send every member a box every time the disease forecast projects high case numbers in a certain area or they may be insufficient boxes to provide everyone with testing supplies, in some embodiments, the insurance provider may be able to adjust the risk profile such that only members with a certain risk score will receive a box. For example, only members with a risk score above a threshold value may receive a box. Determining risk scores for users is discussed in more detail below. In some embodiments, the threshold can be adjusted to match a desired number of test boxes to be shipped or a desired cost.

In some embodiments, the web application may provide a cost estimate of delivering the boxes to all members selected in the Forecast UI. When the insurance provider adjusts the risk profile, the estimated cost of delivering the boxes will increase, decrease, or remain unchanged depending upon changes in the number of users to whom boxes should be sent. In some embodiments, the web application may predict the cost savings associated with the delivery of the boxes. Calculating the cost savings may be based on the web application performing analytics to estimate how many members are likely to need to go to the emergency room (ER) if they contract the infectious disease, how many members are likely to need to stay in the hospital for an extended period of time if they contract the disease, and/or the like. The cost estimate may be based partly on the estimated cost of visiting the ER, estimated cost of a hospital stay, effectiveness of the drug, what percentage of members will take the test and the drug if given the option, and/or the like. The cost estimate may also factor in the members profile to determine whether they are likely to need hospital treatment. For example, members with a high-risk score may be more likely to need hospital treatment than members with a low-risk score. Methods for determining cost savings are discussed in more detail below.

In some embodiments, the Forecast UI may inform the insurance provider which members have already received boxes, when the boxes were received, the estimated expiration date of the boxes currently held by members, the number of tests remaining in member boxes, whether members have used tests in response to infectious disease alerts in the past, and/or the like. Based on this information, the web application may exclude certain members. For example, if a member received a box two months ago, no tests have been used, and the tests have a two-year expiration date, that member may not be selected when determining which members to send boxes to. In some embodiments, members may be able indicate that they do not wish to receive boxes. If members have made this indication, those members may not receive a box. In some embodiments, the insurance provider 130 may specify members who should not receive a box. In some embodiments, the insurance provider 130 may specify one or more criteria for who should be excluded from receiving a box, and the service provider 110 may determine which members meet the criteria.

In some embodiments, the web application may show via the Forecast UI which members have received a box but have not used any tests. The member may not have used a test because, for example, they chose not to in response to an alert, they have not received an alert before, and/or the like. In some embodiments, the Forecast UI may be used to send an informational alert to these members that may, for example, remind them about the boxes they have, provide more information about the tests, provide more information about infectious disease, provide more information about the benefits of test and treatment, and/or the like.

In some embodiments, the Forecast UI may show a variety of metrics associated with an insurance provider's use of the system. For example, the Forecast UI may show how many boxes have been delivered, how many test have been used, what percentage of members who received boxes have used tests in response to the alert, the total cost spend on boxes, the total estimated cost savings based on use of the boxes, and/or the like. In some embodiments, the web application may provide an insurance provider with the option of generating targeted ads promoting the boxes. Use of targeted ads may utilize member data such as zip codes. In some embodiments, the targeted ads may be deployed on one or more social media platforms. In some embodiments, the targeted ads may promote use of the tests by showing the benefits associated with the tests such as, for example, reduced risk of serious health effects resulting from not treating the infectious disease early, reduced costs in not having to go to the hospital, and/or the like. In some embodiments, the targeted ads may alert members of a need for testing. For example, some users may have outdated contact information, may have opted out of (or not opted into) notifications, and so forth. Thus, some members may not be aware that testing may be warranted.

At 408, once the insurance provider has adjusted the risk profile at 406, the insurance provider may schedule the delivery of boxes to the selected members, such as, for example by selecting a “send box button” via the Forecast UI. As described herein, service provider 110 may receive the order for the boxes and member information via the web application and may schedule the orders for the boxes to the selected members. In some embodiments, the web application may provide updates to the insurance provider via that Forecast UI that shows how many boxes are scheduled for delivery, how many boxes have been delivered, percentage of delivered boxes, estimated time to complete the delivery of all boxes, and/or the like.

At 410, the insurance provider may use the Forecast UI to schedule an alert to be delivered by service provider 110. For example, the insurance provider may have delivered boxes in the past, ahead of a projected infectious disease increase in a certain area and may see via the Forecast UI that numbers are likely to increase in a specific area where members are located and have boxes. Based on this information, the insurance provider may choose to send an alert to their members that begins step 108 as described with reference to FIG. 3 above. In some embodiments, the web application may provide alerts or warnings to insurance providers when infectious disease rates are projected to be high in a certain area where the insurance providers members are located.

### Risk Score Determination

As discussed above, insurance providers or other entities can use risk scores in determining which users should receive boxes. This can be done for a variety of reasons. For example, an insurance provider may have limited resources to spend on boxes, there may be a limited number of boxes available, and so forth. Thus, it can be important to target the boxes at those who need them most. Risk scores can also be used for other purposes, such as determining which users should receive greater proactive outreach, such as a phone call, e-mail, text message, app notification, and so forth. While risk scores can provide valuable insight, often such scores are based on limited information that omits a variety of relevant factors. For example, those who live in shared housing or who work in certain jobs (for example, in a restaurant kitchen where multiple people are in close contact with one another, or as wait staff who interact with members of the public in close proximity as part of their job) may be at elevated risk even if their basic health information would indicate that they are relatively low risk. For example, a twenty-year-old with no adverse health history may seem to be at low risk. However, if that same twenty-year-old works in close contact with the public and/or lives in shared housing, they may nonetheless be at relatively high risk of contracting an illness. Conventional analytical approaches may fail to recognize certain risk factors or factors that, when combined with one another, indicate a high risk of contracting an infectious disease and/or suffering significant adverse effects should they contract an infectious disease. For example, workers who earn relatively low wages may not be able to afford to go to a doctor, may not be able to afford to take time off from work to rest and recover, and so forth.

In some embodiments, an artificial intelligence (AI) or machine learning (ML) model can be trained to identify users who are at elevated risk. In some embodiments, the AI/ML model can be configured to determine a risk score for a user. In some embodiments, the AI/ML model can be configured to output one or more risk sub-scores. For example, the AI/ML model can output a risk sub-score based on the user's health history. In some embodiments, the AI/ML model can output a risk sub-score based on the user's living situation (e.g., alone, with roommates, in an apartment, in a single-room occupancy building, in a single-family home, etc.). In some embodiments, the AI/ML model can output a risk sub-score based on the user's mode of transportation. For example, users who drive to work can have a lower transportation risk sub-score than users who rely on public transit such as trains and/or busses.

If sub-scores are used, an overall risk score can be determined using various methods. For example, the overall risk score can be equal to the maximum risk sub-score, can be equal to the average of the risk sub-scores, can be equal to the sum of the risk sub-scores, can be a weighted average of the risk sub-scores, a geometric mean of the risk sub-scores, and so forth.

In some embodiments, risk sub-scores may not be used. For example, if different factors are considered in isolation, combinations of factors that indicate a higher risk may be neglected. Moreover, there can often be missing data about individuals. For example, an individual's health and employment status may be known, but information such as their housing situation may be unknown. Often, humans may be unaware of common associations in data. Moreover, humans may generally not be capable of recognizing some associations. If humans determine the rules for evaluating risk, it is necessary for humans to make inferences about relationships and/or associations. This task can be effectively impossible for relatively rare combinations of data or for complex interplay between factors. However, AI/ML algorithms advantageously do not need to make any such inferences. Thus, an AI/ML model can provide greater predictive insight than would be possible using rules designed by humans.

FIG. 5 depicts a flow chart for training an artificial intelligence or machine learning model according to some embodiments. The process 500 can be run on a computing system. At block 501, the system may receive a dataset that includes user health information such as user surveys, historical test results, genetic information, and/or other medical information (e.g., height, weight, age, etc.). In some embodiments, the dataset can include non-health information such as employment information, income information, transportation information, housing information, distances to pharmacies, distances to healthcare providers, and so forth. At block 502, one or more transformations may be performed on the data. For example, data may require transformations to conform to expected input formats, for example to conform with expected date formatting, units (e.g., pounds vs kilograms, Celsius vs Fahrenheit, inches vs centimeters, etc.), address conventions, and so forth. For example, in some embodiments, addresses can be converted or altered to conform to standards published by the United States Postal Service or a similar postal authority. In some embodiments, the data may undergo conversions to prepare it for use in training an AI or ML algorithm. For example, categorical data may be encoded in a particular manner. Nominal data may be encoded using one-hot encoding, binary encoding, feature hashing, or other suitable encoding methods. Ordinal data may be encoded using ordinal encoding, polynomial encoding, Helmert encoding, and so forth. Numerical data may be normalized, for example by scaling data to a maximum of 1 and a minimum of 0 or −1. These are merely examples, and the skilled artisan will readily appreciate that other transformations are possible. At block 503, the system may create, from the received dataset, training, tuning, and testing/validation datasets. The training dataset 504 may be used during training to determine features for forming a predictive model. The tuning dataset 505 may be used to select final models and to prevent or correct overfitting that may occur during training with the training dataset 504, as the trained model should be generally applicable to a broad spectrum of patients. The testing dataset 506 may be used after training and tuning to evaluate the model. For example, the testing dataset 506 may be used to check if the model is overfitted to the training dataset. The system, in training loop 514, may train the model at block 507 using the training dataset 504. Training may be conducted in a supervised, unsupervised, or partially supervised manner. At 508, the system may evaluate the model according to one or more evaluation criteria. For example, the evaluation may include determining how often the model determines reasonable risk scores for a patient. At 509, the system may determine if the model meets the one or more evaluation criteria. If the model fails evaluation, the system may, at 510, tune the model using the tuning dataset 505, repeating the training 507 and evaluation 508 until the model passes the evaluation at 509. Once the model passes the evaluation at 509, the system may exit the model training loop 514. The testing dataset 506 may be run through the trained model 511 and, at block 512, the system may evaluate the results. If the evaluation fails, at block 513, the system may reenter training loop 514 for additional training and tuning. If the model passes, the system may stop the training process, resulting in a trained model 511. In some embodiments, the training process may be modified. For example, the system may not use a tuning dataset 505. In some embodiments, the model may not use a testing dataset 506.

While described above with respect to determining risk scores, a model can be trained for use in a wide variety of problems. For example, as discussed in more detail below, a model can be trained to determine cost savings, to determine appropriate treatment plans for patients, and so forth.

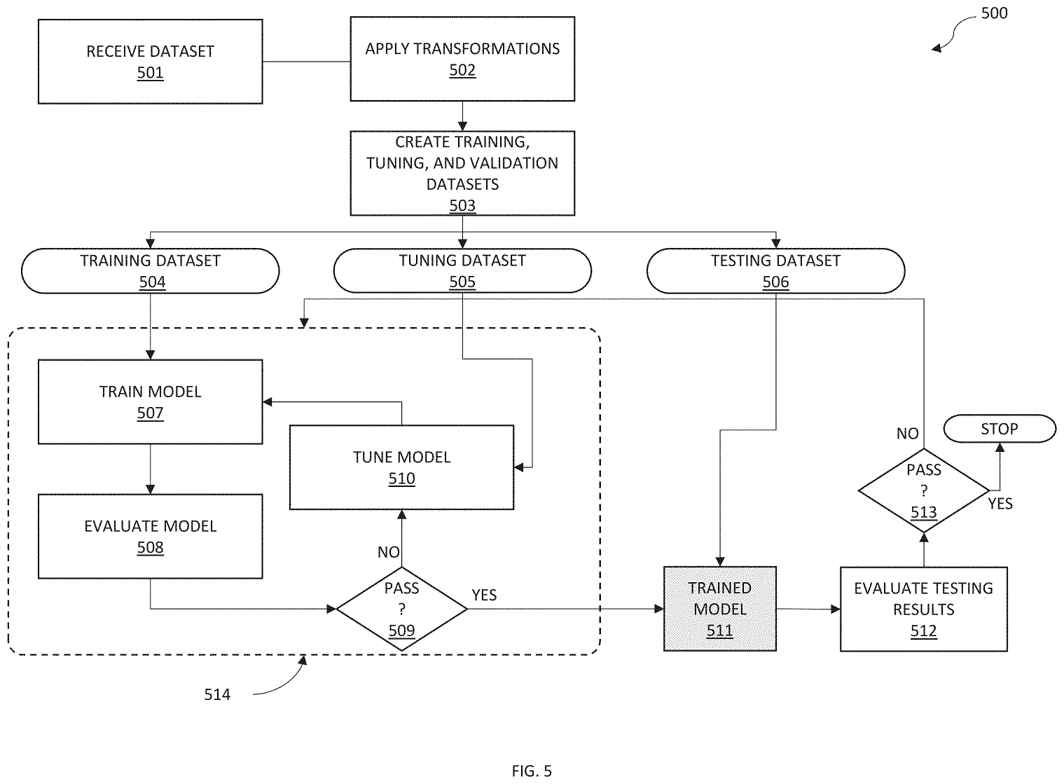

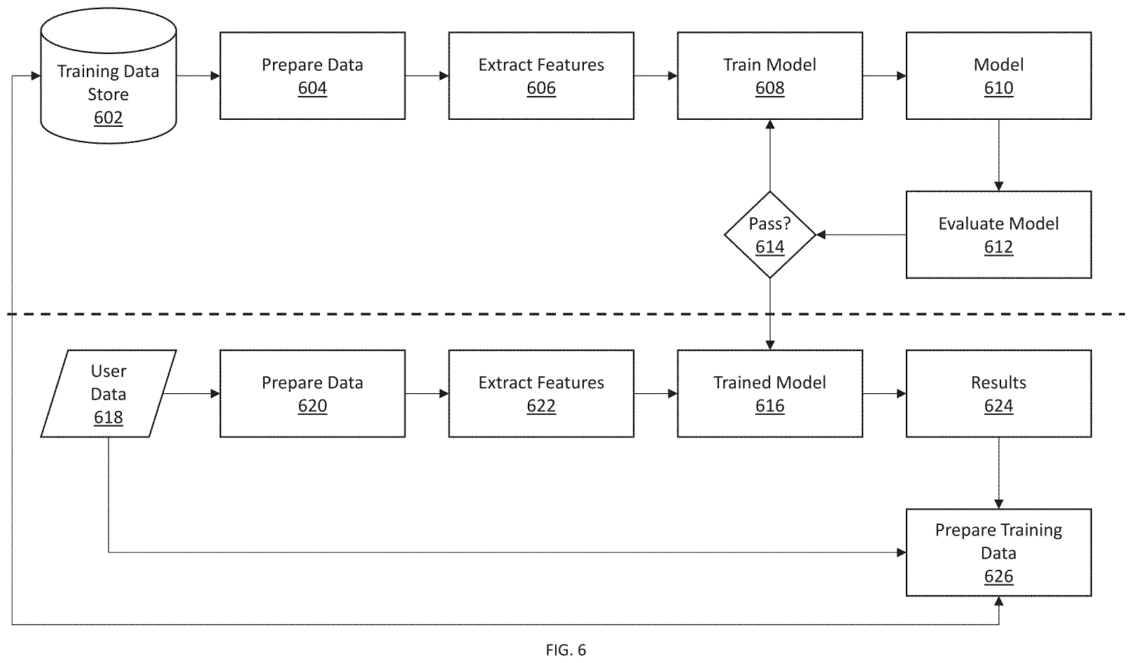

FIG. 6 illustrates an example of training and using an AI/ML model according to some embodiments. The process depicted in FIG. 6 can be used for various purposes, such as to determine risk scores for users, to determine likely costs if a user does not receive at-home treatment, to determine cost savings, to determine treatment plans, and so forth. Training data store 602 can store data for training a model. For example, training data store 602 can store information about users' health, age, socioeconomic status, employment status, housing arrangements, transportation, and so forth. The training data can be annotated to include information about user outcomes. For example, the user outcomes can indicate whether a user had to miss work due to illness, was hospitalized, visited an emergency room, visited an urgent care facility, and so forth. The training data can indicate whether a user received medication to treat an illness at home, treatments delivered at a hospital or other healthcare facility, did not receive any treatment, and so forth. At block 604, a system can be configured to prepare the training data if it was not previously prepared for use in training a model. As described briefly above, preparing the training data can include performing one or more normalization procedures, standardization procedures, and so forth, such as converting units (e.g., between Fahrenheit and Celsius, between inches and centimeters, between pounds and kilograms), converting dates to a standard format, converting times to a standard format, and so forth. In some embodiments, similar treatments or symptoms may be described or coded differently by different healthcare providers. In some embodiments, different providers may use different coding schemes (e.g., ICD-9, ICD-10, ICD-11). Even within a particular coding scheme, providers may select different codes to indicate similar information. For example, ICD-10 has multiple codes for fever, depending upon the origin of the fever (and whether or not the origin is known), when the fever occurred (e.g., post-vaccination, post-procedure, etc.), whether other conditions are associated with the fever, and so forth. A large number of similar codes can lead to variances in coding. Thus, in some embodiments, a code can be changed to another related code. In some cases, certain codes can be excluded if they are not relevant to the issue that the model is intended to address. For example, a patient who presents with COVID-19 may also receive care for an unrelated problem, such as a fractured rib, skin condition, etc., which may not be relevant to the patient's risk of developing severe symptoms. In some embodiments, it can be desirable to exclude certain data as additional data can consume additional computing resources and it can take longer to train a model. However, in some embodiments, exclusions may not be desirable as there can be a risk of excluding a factor that actually is relevant to the patient's risk. In some embodiments, prescribing information may be indicated differently (e.g., a generic drug name or a brand drug name, “BID” or “B.I.D.” or “BD” or “B.D.” to indicate that a medication should be taken twice daily, and so forth). Thus, in some embodiments, data preparation at block 604 can include modifying or removing coding data, treatment data, and so forth. At block 606, the system can extract features from the training data and, at block 608, can train the model using the training data to produce model 610. At block 612, the system can evaluate the model to determine if it passes one or more criteria. At decision point 614, if the model fails, the system can perform additional training. If, at decision point 614, the model passes, the system can make available trained model 616, which can be the model 610 after training is complete.

The trained model 616 can be used to evaluate a particular user. User data 618 can relate to a specific user for whom the outputs of the trained model 616 are desired. At block 620, the system can prepare the data, for example as described above in relations to the stored training data. At block 622, the system can extract features from the prepared user data. The system can be configured to feed the extracted features to the trained model 616 to produce results 624. The results 624 can be used to, for example, determine a risk level associated with the user and/or to determine one or more risk sub-scores for the user.

In some embodiments, the user data 618, the results 624, and other information about the user (e.g., information about the user's outcomes after either receiving or not receiving at-home treatment for an infectious disease) can be used to train the model. At block 626, the system can user prepare the user data 618 and the results 624 for use in training. Preparing the data can include, for example, anonymizing the data. For example, any information about the patient's name, social security number, or other information that could personally identify the patient can be removed. In some embodiments, the system can anonymize the user data 618 in part by altering the user's birthday, for example retaining only the year the user was born (as age is often an important factor in evaluating ask) or the year and month the user was born. In some embodiments, the system can store the prepared data in training data store 602. In some embodiments, the prepared data can be stored, additionally or alternatively, in another database or data store. In some embodiments, the system can retrain the model on periodically, continuously, or whenever an operator indicates to the system that the model should be retrained. Thus, in some embodiments, the trained model 616 can evolve over time, which can result in, for example, improved risk evaluation over time as the model is trained on additional data. Moreover, because infectious diseases often vary over time, it can be important to ensure that the model is trained or retrained using relatively fresh information. For example, in the COVID-19 pandemic, some variants tended to present with worse symptoms, while others were more easily transmissible but tended to have milder symptoms. In some cases, new treatments or vaccines may become available, which can be important to consider when evaluating risk. For example, data from early in the COVID-19 pandemic may be a poor indicator of current risk as antiviral treatments, monoclonal antibodies, and vaccines have become widely available. Traditional epidemiological approaches can struggle to account for rapidly changing situations. Thus, having a machine learning model that can periodically or continuously be trained using new data can increase the speed with which entities such as healthcare providers, insurance companies, and government entities can react to changes. The AI/ML model can also help to identify risks that would otherwise go unnoticed such as, for example, combinations of factors that indicate a higher likelihood of adverse outcomes (e.g., missed work, severe symptoms, hospitalization, death).

As discussed briefly above, a wide variety of factors can be considered in evaluating a user's risk level. FIG. 7 illustrates some examples of data sources that can be used in determining risk scores and/or risk sub-scores. Different and/or additional sources of information can be used, and the data is not limited to the examples shown in FIG. 7. As shown in FIG. 7, user data 618 (which may contain the same or similar information as that stored in training data store 602), can be sourced from a wide variety of sources. Health providers 702 can provide information about the patient's health history, date of birth, height, weight, medications, and so forth. Insurance providers 704 can provide information about the patient's healthcare coverage (which may indicate, for example, that the patient has or does not have access to affordable healthcare). In some cases, information from insurance providers 704 may provide information about the patient's health that was not received from health providers 702. For example, a user may have switched doctors at some point in the past, a healthcare provider may still have some records in only paper format, a healthcare provider may have migrated to a different electronic medical record platform, and so forth, which may make it difficult or impossible to receive some information from the health providers 702. Nonetheless, insurance providers 704 may have information about past health matters which were submitted to insurance for payment. User 706 can provide data such as health history, biographical information, and so forth. For example, a user can register for a testing service and provide certain information (e.g., name, date of birth, medical history, weight, height, etc.) as part of the sign-up process. Government entities 708 can provide information about the user. For example, a national, state, and/or local health authority may have information about the user's vaccination history and any reportable health conditions the patient may have been diagnosed with. Government entities 708 can, additionally or alternatively, provide information about a user's enrollment in health services such as Medicare, Medicaid, and/or other government-run healthcare programs. In some embodiments, government entities may provide information about the user's community, such as overall vaccination rates, work from home rates, mask usage rates, and so forth, which could impact a user's likelihood of contracting an infectious disease or suffering severe adverse effects from the infectious disease.

As discussed above, risk can vary significantly depending on factors other than the user's general health, age, past medical issues, and so forth. Thus, it can be desirable to obtain information about the user from sources that do not provide information that is directly related to health, although some such sources can include some health information. Real estate services 710 can be used to provide information about the patient's living situation. For example, if the patient's address is known, a real estate data company may maintain a database that indicates the type of building (e.g., apartment, single family home, single-room occupancy, dormitory, and so forth), rental or mortgage rates for the building (which can be a proxy for the user's financial resources, for example), condition, age, and so forth, which can impact a user's risk. For example, a user who lives in a building with several roommates and where a kitchen is shared by the residents of multiple units may be at significantly higher risk than a similar individual (e.g., similar age, similar health history) who resides in a single-family home. Credit bureaus 712 can provide information about the user's financial history, which may be indicative of the user's ability to afford medical treatment. For example, a user with little health history (for example, as provided by health providers 702) may nonetheless suffer from significant conditions. A lack of financial resources can mean that a user is likelier to delay or forego treatment. Employment data repositories 714 can provide information about a user's employment status, pay, and so forth. The employment information can indicate, for example, that a user likely has significant contact with the public (e.g., a restaurant waiter), works in crowded conditions (e.g., a kitchen worker), works in areas with poor ventilation and/or poor sanitation practices, and so forth. Such users may be at increased risk for contracting an infectious disease and/or developing significant symptoms. Transportation services 716 can provide information that indicates the user's level of access to public transportation, taxis, car share services, ride hailing services, rental bicycles, and so forth, which can be important as users who rely on mass transit such as busses and trains may be in frequent, close contact with other individuals who may be infected, placing them at higher risk of contracting an infectious disease. On the other hand, users without access to transportation services may be less likely seek out medical care, particularly if they do not live close to a health provider's facility.

As will be appreciated from the above description, user data can come from a wide variety of sources and can relate to a wide range of considerations beyond conventional health factors that relate primarily to information about the patient's body and any conditions, treatments, and so forth. While such data can be valuable in evaluating a patient's risk, the ability of humans to analyze such data is limited. For example, a human being may be able to recognize that older users with significant medical conditions are likelier to suffer adverse events if they contract an infectious disease, but a human could not feasibly determine the many indicators that can contribute to increased risk, particularly when multiple factors interact with one another to create an overall higher or lower risk. On the other hand, an AI/ML model can readily be trained to evaluate risk and can consider the interplay of different factors to create holistic evaluations of a user's risk.

Using data from multiple sources can present significant challenges. For example, there can be mismatches in names, dates of birth, identification number, address, and so forth between data sources. Such mismatches can be because of typographical errors, differences in formatting, differences in how data is collected, a user moving (which may result in addresses not matching), and so forth. For example, one data source may include a user's middle name, which another may include only a middle initial or no middle name at all. In some cases, a user may appear more than once in the same data source. In some embodiments, multiple matches can be returned. For example, if a query is based on a user's name, there may be multiple individuals with the same name.

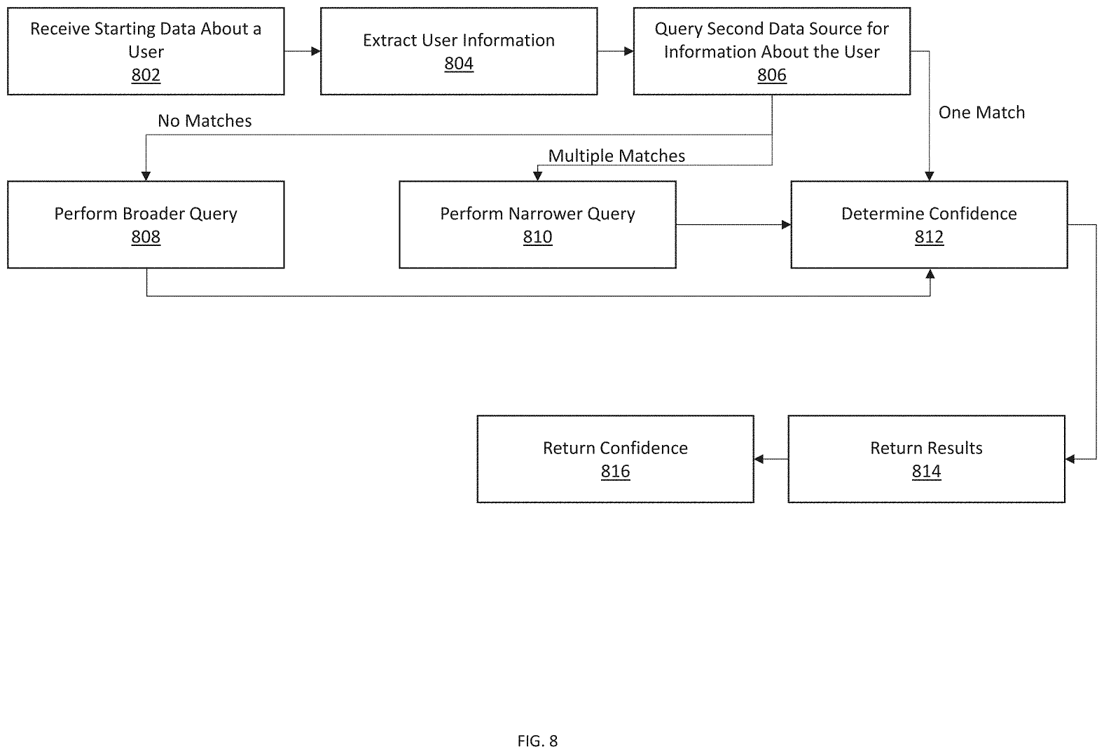

FIG. 8 illustrates an example process for retrieving information corresponding to a user. At block 802, a system can receive starting data about a user. For example, the starting data can be information that the user entered during a sign-up process, information provided by a healthcare provider, information provided by an insurance company, information provided by an employer, and so forth. At block 804, the system can extract user information from the received data. For example, the system can determine the user's first name, last name, social security number (or similar national identification number), driver's license number, address, and so forth. At block 806, the system can query a second data source for information related to the user. For example, the system can query the second data source for information that is specific to the user (e.g., employment information, credit information, and so forth). In some embodiments, the system can query the second source using information such as a zip code or street address, for example to determine how far a healthcare provider is from the user's home, ease of access to a pharmacy, access to transportation, and so forth.

The query can return one record, multiple records, or no records. If the query returns only one record, the system can, at block 812, determine a confidence level that the information is correct for the user. For example, the system can compare fields that were not used to perform the query to determine a likelihood that the record does correspond to the user. For example, if a query were performed using the user's first and last name, the system may compare information such as address, identification number, etc., that is included in both the starting data and the results of the second query. If there are no matches, the system can, at block 808, perform a broader query. For example, the system may include fewer criteria in the query of the second data source and/or may use wildcards and/or fuzzy matching to find potential matching records in the second data source. In some embodiments, such a process can be performed if there was only one match or if there were multiple matches. This may be desirable if, for example, a confidence determined at block 812 is below a threshold level, in which case it may be desirable to retrieve additional results for a broader query, which may have a higher confidence. In some embodiments, if there are multiple matches, the system may perform an additional, narrower query, for example by adding in additional criteria, using no or fewer wildcards, and so forth. The system can, at block 812, determine a confidence level for each record of the broader query at block 808 and/or the narrower query at block 810. In some embodiments, the system may select a query result having the highest confidence. In some embodiments, the system can be configured to provide an interactive experience to an operator, and the operator can review the results (e.g., results with confidence at or above a threshold value). In some embodiments, the operator can select which, if any, result should be used for the patient.

In some embodiments, the system may not determine confidence scores. For example, the system may simply return the results of the second query. In some embodiments, additional queries (e.g., narrower and/or broader queries) may not be performed by the system. While these approaches can result in faster queries and/or lower computational loads, there is a risk that some data for the user may not be located or data for a different individual may be associated with the user.

At block 814, the system can return the results of the second query, which can form part of the user data, for example user data 618 of FIGS. 6 and 7. At block 816, the system can return the confidence value for the result, if such a value were calculated. In some embodiments, the system can store the confidence value as part of the user data. In some embodiments, the confidence value may be used to select which record to incorporate into the user data, but the confidence value may not be retained as part of the user data.

In some embodiments, the system may be configured with a list of rules and/or relations that can be used when querying the second data source. Such information can be stored in a flat file, database table, JSON file, etc. The information can include, for example, rules about date formats, identification number formats (e.g., whether or not dashes are included), storage conventions (e.g., whether the data source stores middle names, middle initials, suffixes, and so forth), corresponding field names (e.g., “first name” in one data source may be called “firstName” in another data source), and/or other information that can aid in crafting well-formed queries that return the desired results. In some embodiments, standards such as HL7 can be used to simplify the sharing of information between different systems that store healthcare data. However, there may not be a similar standard available when retrieving data from other sources.

While illustrated with only a second data source, it will be appreciated that the process described above and illustrated in FIG. 8 can be performed on any number of data sources, such as the data sources shown in FIG. 7.

Information about patients can become stale over time. For example, individuals tend to move, change insurance providers, seek out additional medical treatment, change employers, and so forth. In some embodiments, the system can store a record in the user data indicating the last time the information was retrieved from external sources and/or the last time the user updated or confirmed information supplied by the user. In some embodiments, the system can be configured to periodically check for updated information, for example by performing new queries on the additional data sources, by prompting the user to update or confirm information, and so forth. For example, a user may be prompted to update their information monthly, quarterly, twice per year, annually, or at any other frequency as may be desired. In some embodiments, the system can be configured to query the data sources whenever the user logs in, whenever the user is to take a new test, whenever infections in the user's location are increasing, and so forth.

### Post-Treatment Follow-Up

In some cases, follow-up care may be warranted after a user takes an at-home test or receives a treatment. For example, the follow-up care can include determining if the user's symptoms have improved, determining if additional treatment may be warranted, and so forth. Accordingly, in some embodiments, a user can sign up for follow-up services. In some embodiments, the user may be assigned a risk score as described above. In some embodiments, the risk score can be used in determining a follow-up strategy and/or whether follow-up is warranted.

A user may undergo (e.g., self-administer) a medical diagnostic test with the aid of a remote medical diagnostic testing platform. In some embodiments, the user may purchase a test at a pharmacy or other retailer. In some embodiments, the test can be provided by the user's insurance, healthcare provider, and so forth. The testing platform may guide the user though performing the test and interpreting the results. In some embodiments, such guidance is provided via a proctor associated with the testing platform. Once the user receives his or her test results, one or more post-test steps may need to be taken. For example, a user may be prescribed a treatment based on the results of the test.

To this end, a follow-up service may be initiated after the administration of a health or diagnostic test. The follow-up service may be provided to the user to assist the user in navigating a recovery process, a treatment process, or the like.

In some embodiments, the user may sign up, opt into, or otherwise be enrolled in a follow-up service after the administration of a health or diagnostic test. In some embodiments, a user may be automatically enrolled in a follow-up service. in some embodiments, a user may be automatically enrolled in a follow-up service only if the user has a positive test result. A doctor, healthcare provider, and/or the testing platform may provide user personal information to a system and determine a follow-up interaction. In some embodiments, the follow-up service may then contact the user. In some embodiments, the follow-up service may be subscription-based. In some embodiments, the follow-up service may allow treatment or healthcare providers to inquire about the user's symptoms, subjective or objective feelings, medication plan, overall status, or the like. In this way, continued care can be provided to the user.

The follow-up service can be instantiated after the user undergoes a medical diagnostic test. In some instances, the user may perform the test using a remote testing platform that guides the user through performing the test and interpreting the results. The user may receive or be prescribed a treatment based on the results of the administered test. Such treatment may be provided by a doctor, physician, or other qualified professional. After the user administers the test, the user may sign up for or be enrolled in a follow-up service. In some embodiments, the user may be automatically enrolled in the follow-up service, a physician may enroll the user in the follow-up service themselves, a third-party provider may enroll the user in the follow-up service, and/or the testing platform may enroll the user in the follow-up service. Such third-party providers may include, for example, an insurance company, health maintenance organization (HMO), healthcare provider, and so forth. In some embodiments, follow-up services may be provided on a subscription basis. The subscription basis may be weekly, monthly, yearly, etc.

In some embodiments, the follow-up service may be used by and benefit more than just the user. For example, the follow-up service may be used or implemented by doctors' offices, hospitals, insurance companies, healthcare providers, medical clinics, etc. Such parties can use the follow-up service to improve adherence to prescribed treatments, monitor users after office or online visits, and/or the like.

In some embodiments, to enroll a user in the follow-up service, a provider may provide information to the service. This information may include the user's name, medication prescribed, medication schedule, user contact information, etc. In some embodiments, the provider can, at the same time or later, select a level of follow-up interaction based on the doctor's or provider's analysis or judgment of the treatment, the user's preference, or both. Various levels or types of follow-up interactions are possible and can include, for example, daily, weekly, or monthly follow ups, follow ups based on times that medications should be taken, etc. The follow up can be provided in various ways including sending a text message, email, notification, reminder, or other communications. In some embodiments, the level of follow-up can be determined at least in part based on a risk score assigned to the patient, as described above.

In some instances, through the follow-up service, the user may be prompted to answer follow-up questions. The follow-up questions may include, for example, symptom checkers, overall subjective feeling questions, questions regarding whether the user has a healthcare provider, whether the user had difficulty obtaining their prescription, whether the user has been taking their medication regularly as prescribed, etc. In some instances, depending on the user's answers to the follow-up questions, treatment or healthcare providers may be prompted to contact the user. In some instances, with contacting the user, the provider may request the user schedule a follow-up appointment, send additional testing supplies to the user, and/or instill other follow-up procedures. The provider may request the user to schedule a follow-up appointment by directing the user to a scheduling site, by having office staff further contact the user to set up an appointment, or other modes of scheduling appointments. For example, additional testing supplies can include the same type of test the user previously administered, a different type of test than the user previous administers, or other supplies the user may need. In some instances, the user may be sent a different test to determine whether there are any outstanding or additional medical issues present. In some embodiments, the user may be sent the same test again, for example to determine if the user still tests positive for a particular illness, still has a particular vitamin deficiency, and so forth.

In some instances, the follow-up service may collect user data over time. This data may be objective and/or subjective data that can be collected during the follow-up process. For example, objective data may include whether the user received treatment. The user data may be populated into a database that may allow for user or physician viewing. In some instances, the user may be asked to opt in to being contacted by a provider to collect the user specific data. In some instances, this may be regardless of whether treatment was provided to the user. Additionally, the data collected may be anonymized or user specific. In some embodiments, the follow-up service can collect testing data to determine if a user tests positive or negative for an infectious disease over time. In some embodiments, the follow-up service can collect testing data over time to determine if a user's vitamin deficiency has been resolved, if a user's allergy medication is working, and so forth.

In some instances, the follow-up service may differentiate between users that received treatment and users that did not. For example, the follow-up services for users that did not receive treatment may include suggesting an additional test. The additional test may be used to confirm the results of the original test. In some instances, the additional test may be a different test than the test the user previously administered. For example, a user who has previously taken a COVID-19 test may take an additional flu test, or a user who took a rapid COVID-19 test may take a PCR test, which may provide more reliable results. Furthermore, the follow-up service for users that did not receive treatment may include suggesting general home remedies, over the counter solutions related to the user's symptoms, check-ins with the user over the subsequent days, weeks, and/or months to assess improvement in symptoms, etc.

In some instances, the follow-up service may be configured to collect experience data. The experience data may include data regarding the user's experience with the follow-up service. In some instances, the user may be asked to opt into future contact with the follow-up service. For example, the experience data can be used to determine how users felt about the number of follow-up contacts, the timing of follow-up contacts, the quality of follow-up care, and so forth.

Assisting the user in navigating the recovery process may be important. Accordingly, it may be beneficial to provide the user with a follow-up service. This may allow the user to navigate the recovery process in a structured and cared for manner. For example, in some cases, the follow-up service may include communications with treatment providers.

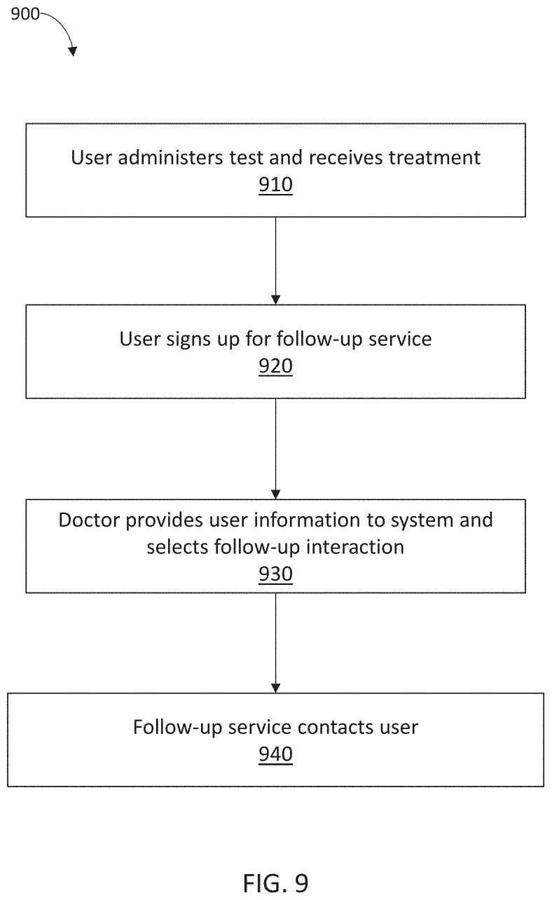

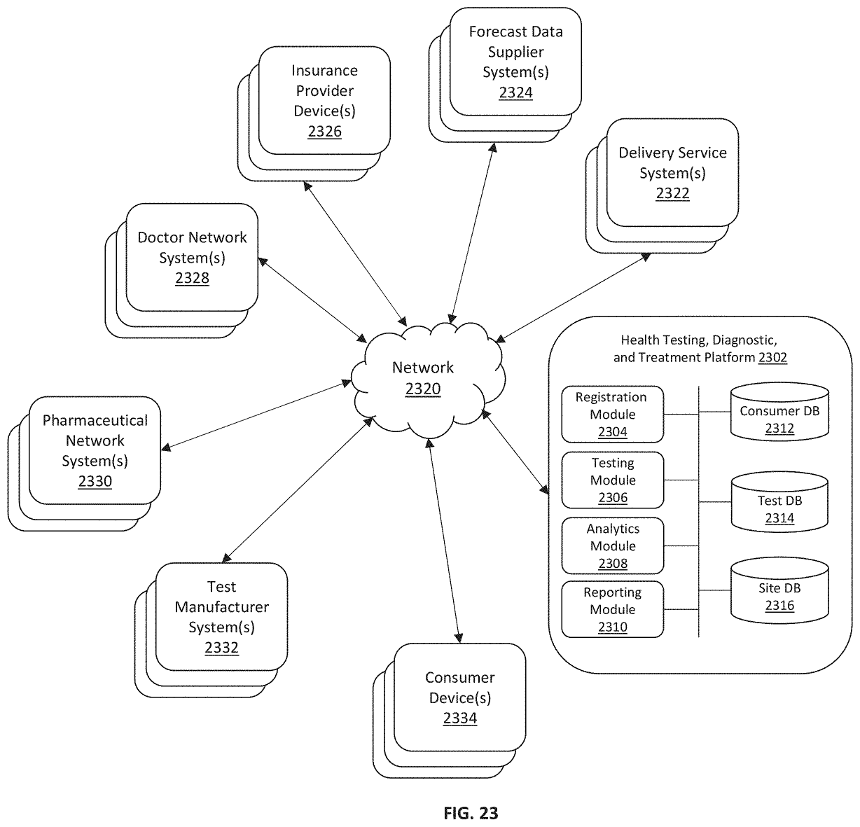

FIG. 9 is a block diagram illustrating an example follow-up service protocol or method. The method 900 can be implemented, for example, using one or more components of the system shown in FIG. 23.

At block 910, the user may administer a test and receive treatment pertaining to the results of the administered test. The test may be a diagnostic or health test that may be administered remotely. At block 920, the user may sign up or opt into a follow-up service. The follow-up service may be provided on a subscription basis. The follow-up service may be used by users, doctors' offices, hospitals, insurance companies, etc., as a method to improve adherence to prescribed treatments and monitor users after in-office, remote, or online visits or communications. The follow-up service may include the user being prompted to answer questions, which may inquire about symptoms, overall subjective feelings, ability to obtain prescriptions, and medication administration.

At block 930, a doctor or healthcare provider may provide a system with user personal information. The user personal information may include the user's name, medication prescribed, medication schedule, contact information, etc. The doctor or healthcare provider may also select follow-up interaction, which may be based on the doctor's analysis or judgment, the user's preferences, a risk score assigned to the user, or any combination of these considerations. This may determine the frequency of which the user is followed up with.

At block 940, the follow-up service may contact the user. In contacting the user, the follow-up service may prompt treatment providers to call the user, request that the user schedule a follow-up appointment, send additional testing supplies, inquire how the user is doing, inquire whether the user was able to receive their medication, inquire about the user's use of the medication, inquire about any side effects or changes in the user's condition, etc. For example, a user who lacks reliable transportation and doesn't live near a pharmacy may have trouble obtaining medication, in which case the user may be presented with options for acquiring the medication through, for example, a mail order pharmacy or a local pharmacy that offers delivery services. The follow-up service may contact the user intermittently or consistently via email, phone, text message, instant message, direct message, mail, etc. Additionally, the follow-up service may provide the user with a proctored treatment or medication service wherein a proctor may guide the user through their treatment or medication plan.

### Ongoing Testing and Treatment

While the discussion above relates primarily to testing for infectious diseases, it will be appreciated that the disclosure is not so limited. Embodiments herein can be used for ongoing monitoring of a wide variety of physiological factors and for providing treatment using prescription medications, non-prescription medications, vitamin supplements, mineral supplements, and so forth. While a typical acute illness scenario may involve only one or a few follow-up encounters, some users may benefit from additional follow-up. In some cases, users can receive follow-up care for chronic or long-term issues such as allergies or vitamin or mineral deficiencies. Accordingly, in some embodiments, systems and methods can provide for ongoing monitoring and treatment of users. In some embodiments, the systems and methods herein can be used to adjust a user's treatment regimen in response to test results. For example, a user who does not respond well (e.g., lack of improvement, excessive side effects, or both) to one antiviral medication may be prescribed a different antiviral medication. As another example, a user who has a vitamin deficiency may show improvement after taking a supplement or making changes to the user's diet, in which case the supplement may be reduced in dosage or discontinued.

Often, physicians and other healthcare providers can struggle to adjust a user's treatment plan over time, particularly when multiple treatments are involved. For example, a user who suffers from allergies may take one or more medications. To limit side effects, it is desirable to provide minimal effective doses to the user. However, physicians operating without the aid of an ongoing testing and monitoring platform may struggle to recognize patterns, may face difficulty determining which of several medications to adjust, and so forth. Thus, there is a need for a platform that can provide guidance based on the user's actual test results and actual experiences with a treatment regimen.

Advantageously, in some embodiments, a platform can utilize artificial learning or machine intelligence in determining modifications to a treatment plan. The AI/ML model can be trained as described herein. For example, training data can include information about patient experiences (response to treatment, side effects, etc.), treatment regimens, comorbidities, other medications the patient is taking, and so forth. After training, the model may be provided with information about a patient and may provide various outputs, such as recommended treatment adjustments, estimates of the likelihood the patient will experience significant side effects, and so forth.

### Physiological Testing and Treatment

In some embodiments of a physiological testing and treatment system, a user may perform a physiological test, such as a medical diagnostic test. In some embodiments, the physiological test may be a blood test, a urine test, or any other physiological test. In some embodiments, the test may be performed to test for a level of a physiological indicator such as vitamins, minerals, electrolytes, hemoglobin, blood oxygen level, hydration, blood cell count, glucose, food intolerance, allergies, or any other physiological indicator. In some embodiments, the user may take a panel to test levels for multiple physiological indicators at one time.

In some embodiments, the physiological test may be facilitated over a remote proctoring system. In some embodiments, the user may access the remote proctoring system with a computing device. In some embodiments, the user device may be a mobile computing device. In some embodiments, the remote proctoring system may comprise an application. In some embodiments, the user device may establish a connection with a proctor device. In some embodiments, the connection may allow a proctor or the remote proctoring system to observe or guide the user taking the physiological test. In some embodiments, the remote proctoring system may use augmented reality or virtual reality in order to guide or observe the user taking the physiological test.

In some embodiments, the physiological test may provide a test result after the user completes the physiological test. In some embodiments, the test result may include a user's level of a physiological indicator. In some embodiments, the user's level of the physiological indicator may be less than or greater than an expected level. In some embodiments, based on the user's level of the physiological indicator, the computer device, the remote proctoring system, the application, and/or the proctor may recommend the user at least one treatment (e.g., the proctor may consider the output of a model in determining a treatment). In some embodiments, the at least one treatment may include dietary changes, lifestyle changes, vitamins, supplements, a prescription, powders, food-based ingredients, pre-made meals, fresh food, frozen food, perishable food, non-perishable food, nutraceuticals, or any other treatment.

In some embodiments, the user may meet with a dietician, nutritionist, or other medical professional. In some embodiments, the user may meet with the dietician, nutritionist, or other medical professional before or after the user receives the at least one treatment. In some embodiments, the user may meet with the dietician, nutritionist, or other medical professional multiple times over a period of time. In some embodiments, the user may meet with the dietician, nutritionist, or other medical professional in person or over a network. In some embodiments, the network may include a phone call, a video call, or telemedicine call.

In some embodiments, the dietician, nutritionist, or other medical professional may ask the user questions or provide information related to the test result. In some embodiments, the dietician, nutritionist, or other medical professional may provide the user the at least one treatment. In some embodiments, the dietician, nutritionist, or other medical professional may provide the user the at least one treatment based on the test result and/or information provided by the user in person or over the network.

In some embodiments, the dietician, nutritionist, or other medical professional may enter the information provided by the user into the physiological testing and treatment system. In some embodiments, the user may directly enter information, for example via a web form, mobile application, and so forth. In some embodiments, the physiological testing and treatment system may provide the at least one treatment based on the test result and the information provided by the user.

In some embodiments, the physiological testing and treatment system may deliver the at least one treatment to the user through mail, courier services, email, text message, an application, a web page, or any other delivery method or system. In some embodiments, the physiological testing and treatment system may order the at least one treatment from a third party.

The physiological testing and treatment system may be used for a wide variety of conditions where testing and treatment can be done remotely. For example, a user with diabetes may test for glucose levels and be treated with insulin or sugar as appropriate. An individual with anemia may test for iron, ferritin, transferrin, and/or total iron-binding capacity (TIBC) and be treated with an iron supplement. A user with an electrolyte imbalance may test for one or more electrolyte levels and may be treated with a supplement. Similarly, a user may measure vitamin levels and be treated with a supplement if there is a deficiency. In some embodiments, a user may have allergies or food sensitivities and may take a test to determine allergic response. The user may be treated with an antihistamine or other appropriate treatment. In some embodiments, other tests may also be used, such as tests of the user's environment to determine levels of allergens. In some embodiments, a user may test for inflammation and may receive treatment in response to the test result, such as a non-steroidal anti-inflammatory drug (NSAID).

In some embodiments, the treatment may include a treatment plan. The treatment plan may include scheduling one or more additional tests spread out over a period of time, and/or one or more additional orders of the at least one treatment. In some embodiments, the treatment plan may include a plan for maintaining the user's level of the physiological indicator. In some embodiments, the treatment plan may include a plan to decrease or increase the user's level of the physiological indicator so that the user's level of the physiological indicator is at or about the expected level.

As just one example, the physiological testing and treatment system may be used to optimize vitamin levels. For example, a user may take a test to determine the levels of one or more vitamins. The user may take an initial test and a treatment plan may be developed. The treatment plan may include, for example, a supplement pill, a powder (for example, for adding a drink, smoothie, etc.), and/or nutritional advice. In some embodiments, the physiological testing and treatment system provider may partner with smoothie companies, meal services, and so forth to provide tailored orders to the user.

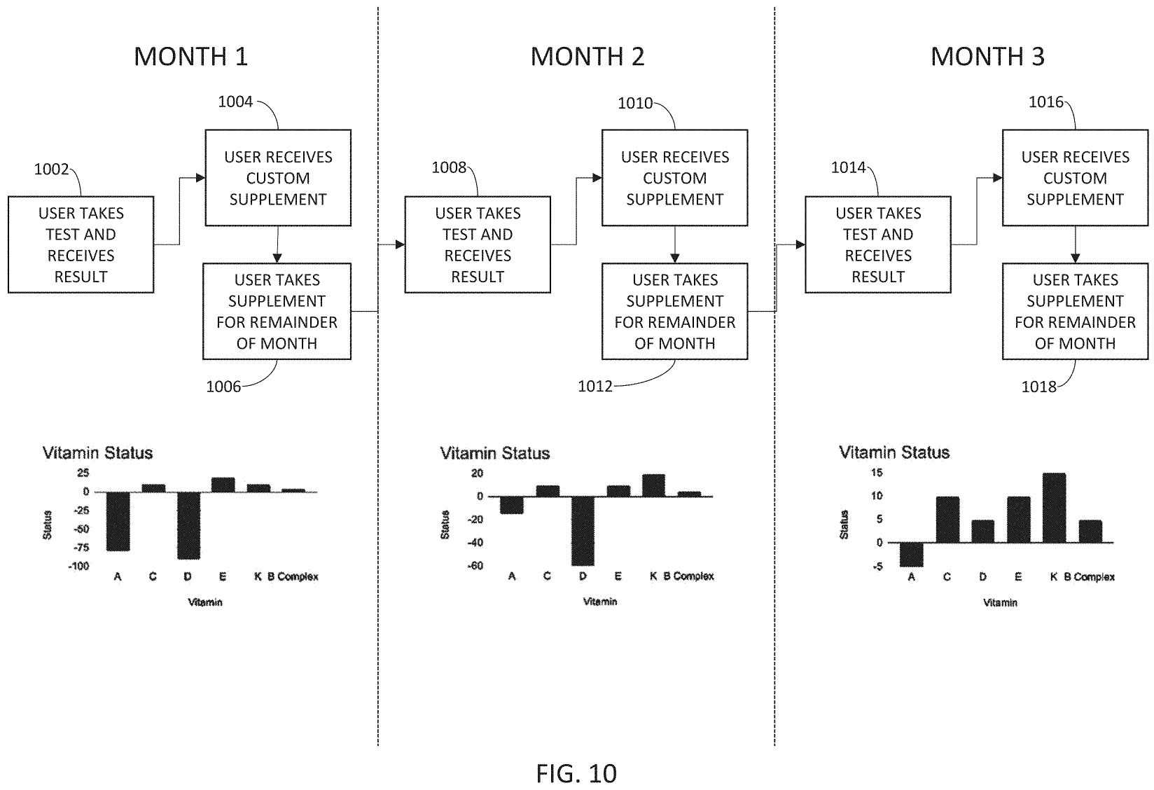

FIG. 10 depicts an example testing and treatment process according to some embodiments. During Month 1, the user takes a test and receives results at block 1002, which may indicate that one or more vitamin levels is outside a normal range. For example, vitamin levels may be measured compared to a target value. As shown in FIG. 10, the user initially has low levels of vitamins A and D, while other vitamins have deviations from ideal values that are within a threshold value of zero. Based on the results of the Month 1 testing, the user receives a custom supplement at block 1004 and takes the custom supplement at block 1006 before taking another test in Month 2 at block 1008. In some embodiments, the user may continue taking the supplement after taking the test in Month 2. For example, the user may take the current supplement until receiving an updated supplement. The Month 2 testing results may indicate changes relative to the Month 1 result. For example, as shown in FIG. 10, the supplement has improved the user's vitamin A level, but the user's vitamin D level is still low. The user may receive an updated supplement based on the Month 2 testing results at block 1010 and may take the supplement at block 1012 followed by another test during Month 3 at block 1014. The Month 3 result may indicate the need for one or more adjustments or, as shown in FIG. 10, may indicate that all vitamin levels are within a threshold range, in which case the supplement may not be modified. The receive can receive a modified supplement or the same supplement at block 1016 and take the supplement at block 1018, for example until the end of the month, for a certain number of days, or until a new test and supplement are taken and received. While monitoring and adjustment is shown as occurring monthly in FIG. 10, other timings are possible. For example, in some cases, more frequent monitoring may be warranted, while in others, quarterly, biannual, or yearly monitoring may be sufficient. Such monitoring and adjusting as depicted in FIG. 10 can be carried out over a short term, such as a few weeks, a few months, and so forth, or can be carried out on an ongoing basis over a long period of time. In some embodiments, monitoring may cease once target vitamin levels are achieved, while in other cases, a user may continue monitoring. In some embodiments, the user may take a test and the supplement may be adjusted in response to certain events, such as changes in the user's diet or exercise.

In some embodiments, the physiological testing and treatment system may monitor persistent or chronic conditions and acute conditions simultaneously. For example, the system may be used to manage seasonal allergies, which may include treatment with one or more medications. A user who suffers from seasonal allergies may take a first drug (e.g., Drug A) to manage symptoms and may take a second drug (e.g., Drug B) as needed for flare-ups of severe symptoms. By monitoring the user's allergic response, the physiological testing and treatment system may determine if a user's treatment regimen should be adjusted to better manage symptoms. Alterations to the user's treatment plan may be stored for future use. Ongoing monitoring and adjustment can have many advantages. For example, users may have fewer flare-ups and/or dosages may be reduced so that the user's side effects are minimized.

Advantageously, monitoring data can be analyzed automatically by a computer system to recommend modifications to a user's treatment. For example, the system can estimate the impact of altering a dosage, discontinuing a medication or supplement, or adding a medication or supplement. In some embodiments, the system can be configured to consider data of other users in estimating impacts. For example, in some embodiments, the system may use regressions based on data from a plurality of users. In some embodiments, an AI/ML model can be trained as described above and configured to output information for adjusting a treatment plan.

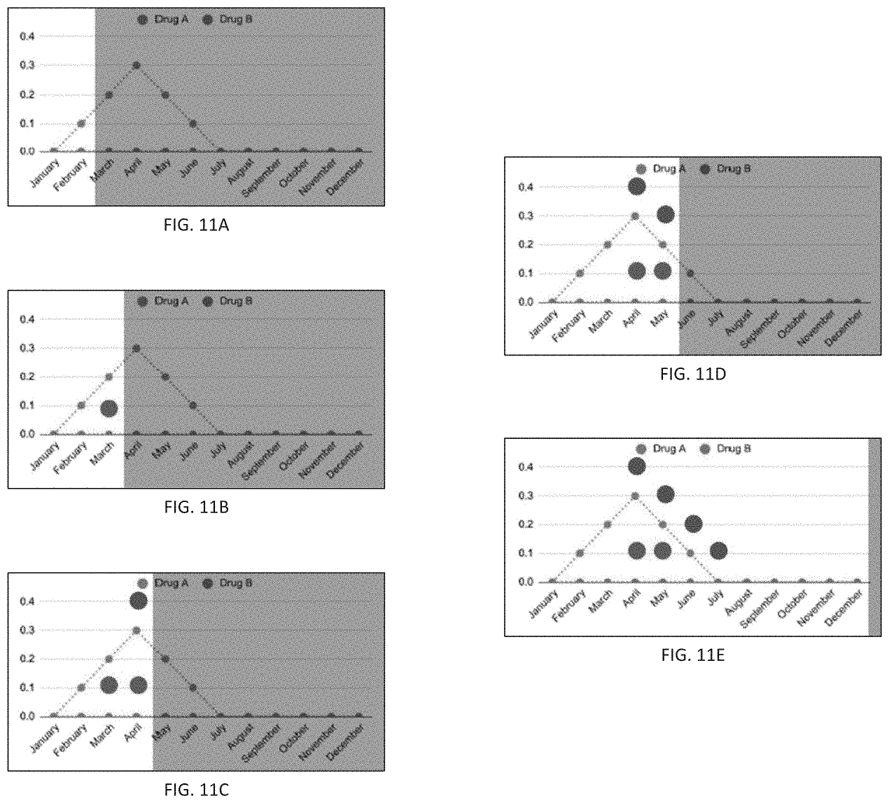

FIGS. 11A-11E depict an example monitoring and treatment adjustment process that may be implemented by the physiological testing and treatment system according to some embodiments. As depicted in FIG. 11A, a user may have a starting treatment plan that may include Drug A at a varying dosage for seasonal allergies (for example, the user may take a higher dose during the peak of allergy season) and Drug B for use as needed to address flare-ups of symptoms. In FIG. 11A, the user may undergo allergy testing in January and February, and allergic responses may be normal, resulting in no changes being made to the user's treatment plan. In March, as shown in FIG. 11B, the user may undergo testing and an allergic response may be detected. In response to detecting the allergic response, the system may recommend that Drug B be used to manage symptoms. In April, as shown in FIG. 11C, the user may undergo testing and may again show allergic response. The system may again recommend Drug B and may also increase the dosage of Drug A. The increase in the dosage of Drug A may be stored by the system and used for developing a treatment plan for the next year. In May, as shown in FIG. 11D, the user's allergic response may be normal. The system may recommend stopping treatment with Drug B while maintaining the increased dosage of Drug A. Over the rest of the year, the user may continue to undergo testing as depicted in FIG. 11E, which may show normal allergic responses. For example, the dosage may be tapered off as the cause of the seasonal allergies abates (e.g., due to changing seasons).

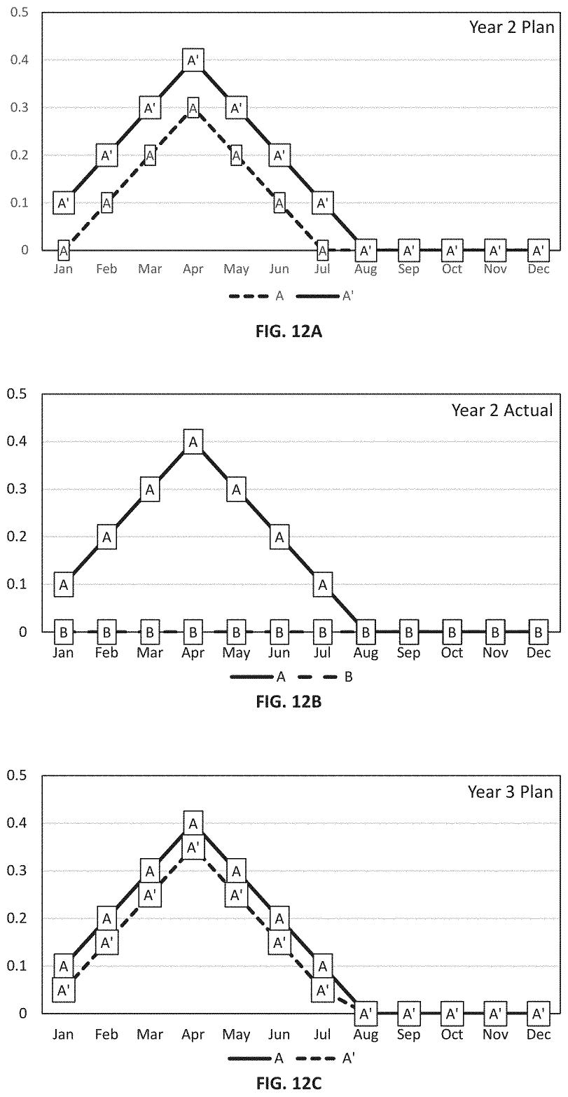

FIGS. 12A-12C depict seasonal allergy management according to some embodiments. Seasonal allergy management may be implemented by the physiological testing and treatment system. As shown in FIG. 12A, a patient's Year 2 plan may be adjusted based on the previous year (e.g., as monitored and adjusted as shown in FIGS. 11A-11E). As shown in FIG. 12A, based on the previous year, the patient's second year may be modified from the original treatment plan (A) to a new treatment plan at a higher dose (A′), for example because the patient still had significant symptoms while taking the first year dose. As the patient proceeds through the second year, they may undergo repeated testing and their dosage may be adjusted. In some cases, as shown in FIG. 12B, the patient may exhibit normal allergic response throughout the year, in which case the patient may not need to use Drug B. This may indicate that the patient's dosage of Drug A can be reduced without worsening symptoms. Thus, in Year 3, as shown in FIG. 12C, the patient's dosage of Drug A (labeled A′) may be reduced compared to Year 2 (labeled A), such that it is in between the Year 1 dosage and the Year 2 dosage. The patient's treatment regimen can thus be optimized both within a season and from season to season or year to year to better manage symptoms.

FIGS. 11A-11E and 12A-12C start from an initial treatment plan and adjust the plan over time. However, some patients may not have an existing treatment plan. Thus, an allergy test such as a comprehensive panel may be performed and used as a starting point for creating a treatment plan. In some cases, the testing may be performed at home and may be part of an overall product for monitoring and managing the patient's allergies. In some embodiments, the patient may regularly receive testing kits, for example as part of a subscription service. In some embodiments, surveys may be used along with testing to gather information from patients. Surveys may ask patients about their conditions, symptoms, and so forth. In some embodiments, the physiological testing and treatment system may use survey data to refine treatment plans. For example, if a patient indicates that they have moved to a new region or that they are spending more time outside, the patient's treatment plan may be adjusted. In some embodiments, the system may also consider environmental testing results. For example, the patient may collect air or water samples from their environment to monitor for allergens, pollution, particulates, and so forth that may cause a reaction in the patient.

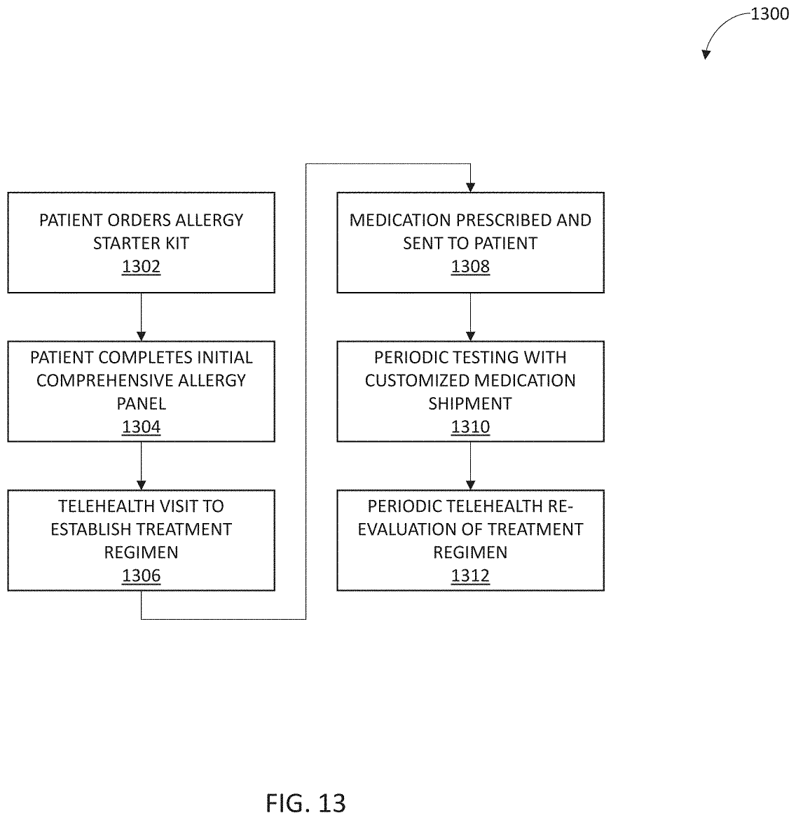

FIG. 13 depicts an example process 1300 for remote testing, treatment, and monitoring according to some embodiments. At block 1302, a user may order an allergy starter kit. At block 1304, the user may use the kit to test to complete a comprehensive allergy panel at home. The kit may provide results directly to the user, or the user may send the kit to a suitable laboratory for processing. At block 1306, the user may engage in a telehealth visit with a provider to establish a treatment regimen based on the results of the allergy panel. At block 1308, the provider may prescribe one or more medications to the user, and the user may receive the medications. In some cases, the medication may be mailed, delivered by courier, or picked up from a local pharmacy. At block 1310, the user may undergo periodic (e.g., monthly, quarterly, or annually) testing and the user's medication may be customized based on the results of the periodic testing. At block 1312, the user may periodically have a telehealth meeting with a provider to re-evaluate the treatment regimen.

While FIGS. 10-13 present vitamin deficiency and allergy treatment scenarios, it will be appreciated that similar techniques can be applied to other conditions that benefit from long-term monitoring and adjustment of treatment levels, such as diabetes, high blood pressure, high cholesterol, and other chronic or long-term conditions. The ongoing monitoring and adjustment techniques herein can be especially valuable in cases where multiple drugs are used to treat a single condition, where a patient suffers from multiple conditions, and so forth. These techniques can be used to ensure that patients receive effective treatment while limiting the potential for excessive treatment, which may trigger drug interactions, side effects, and so forth that can be harmful to the patient. In some embodiments, treatment compliance can be improved if, for example, a patient takes lower dosages and thus incurs fewer side effects, or if a patient is able to limit the number of medications. Simpler treatment regimens can make compliance significantly easier. For example, medications often have different, sometimes conflicting instructions. Some may be taken at night, in the morning, at lunch, after a meal, before a meal, on an empty stomach, and so forth. This can create confusion for patients and lead to missed doses, incorrectly taken doses, and so forth.

In some embodiments, the physiological testing and treatment system may include a dashboard or patient interface displayed on a patient computing device. In some embodiments, the physiological testing and treatment system may include a dashboard or patient interface displayed on a medical professional computing device. In some embodiments, the dashboard may display the patient's level of one or more physiological indicators. In some embodiments, the dashboard may display the patient's level of one or more physiological indicators over a period of time. In some embodiments, the period of time may include one or more physiological tests. In some embodiments, the dashboard may include a graph of the patient's level of one or more physiological indicators over the period of time. In some embodiments, the dashboard may display any information relevant to the patient's physiological tests.

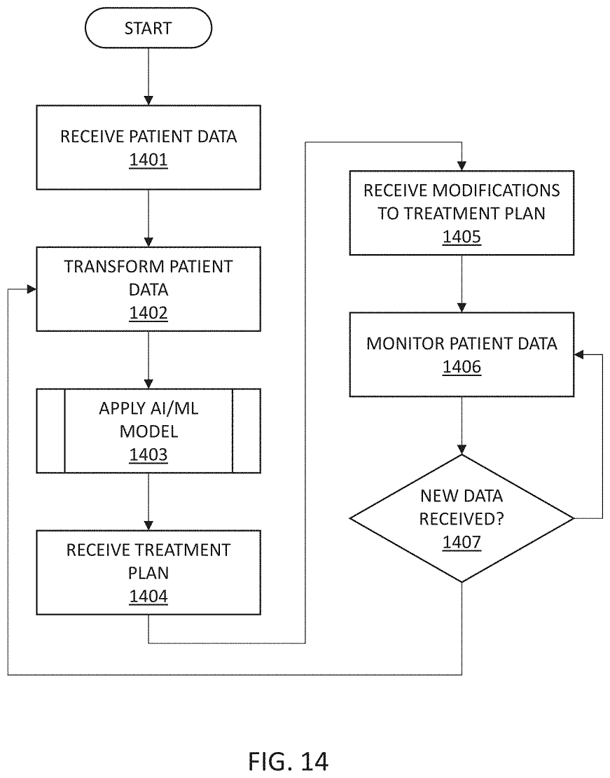

FIG. 14 is a flow chart that illustrates a process that may be run on a computing system for determining a treatment plan and monitoring user performance according to some embodiments. At block 1401, the system may receive user data such as test results or other data. At block 1402, the system may transform the data in preparation for applying a machine learning or artificial intelligence model. At block 1403, the transformed data may be provided to an AI/ML model and, at block 1404, the system may receive a treatment plan from the AI/ML model. In some embodiments, the AI/ML model may not provide a treatment plan but may instead provide information indicative of a treatment plan, which the system may use to determine the treatment plan. At block 1405, the system may receive modifications to the treatment plan. For example, a physician may wish to modify the treatment plan suggested by the AI/ML model. At block 1406, the system may monitor a database or other data source to determine if there is new information about the user. If there is new user data, then at block 1407, the system may re-determine the treatment plan by returning to block 1402. If the system does not detect any new user data, the system may continue to monitor for new user data. In some embodiments, rather than determining a new treatment plan whenever there is new user data, the system may be configured to determine a new treatment plan at specific times, such as when the user has an upcoming appointment or when a physician manually triggers an update of the user's treatment plan. In some embodiments, the AI/ML model may undergo continuous training. For example, the model may be updated as additional health data becomes available, thereby enabling the AI/ML model to produce improved results over time.

### Clinician Portal

In some embodiments, a clinician portal may be provided by the physiological testing and treatment system (or by other systems that provide testing and treatment, medical record access, or other similar functionality). The clinician portal may allow a clinician to see user data, user surveys, historical test results, and so forth. The clinician portal can have a variety of tools that allow a healthcare professional to set alerts based on various conditions, such as a user being due for a test, a new test result being available, and so forth.

In some embodiments, the clinician portal may have a significant amount of data associated with many different users. In some embodiments, a system can leverage this user data for machine learning. For example, machine learning models can be trained to identify users that are at higher risk for certain conditions, higher risk of suffering significant adverse effects of a condition, and so forth. In some embodiments, an AI/ML model can be trained as described above, considering patient health history and/or other factors. In some embodiments, machine learning models may be deployed to automatically flag users that may benefit from clinician follow up based on a combination of data such as lab results, conformance to treatment plans (as many be determined by, for example, user self-reporting or from pharmacy information such as how often the user refills a prescription). In some embodiments, a user's genetic profile may be loaded (e.g., automatically or manually via the clinician portal) and may be used to predict potential health issues. For example, certain genetic mutations or other genetic markers may indicate a predisposition towards certain diseases. For example, certain single nucleotide polymorphisms can indicate an increased risk of certain cancers, celiac disease, macular degeneration, Parkinson's disease, and medical conditions. A clinician may use these predictions to provide education, preventative care, early diagnostic testing, treatment, and so forth. In some embodiments, the machine learning model may determine previously unknown indications of health risks. For example, the AI/ML model may recognize complex data correlations that would be infeasible for a clinician to uncover on their own.

In some embodiments, the system may use a combination of demographic information, medical history information, and/or genetic predisposition information to serve as a starting point for determining a clinician's prescribed treatment protocol. This information can be used in regressions, AI/ML models, and so forth as described herein. This information may be used, for example, to determine recommended ideal levels of vitamins, minerals, blood sugar, hormones, and other physiological indicators for a particular user. In some embodiments, the clinician portal may provide toggles, sliders, or other adjustment mechanisms so that a provider can modify the ideal levels of one or more physiological indicators. In some embodiments, the ideal levels for each physiological indicator can be automatically suggested, for example by using a reference or guideline or by interpolating from a stored library, based on other user information (for example, height, weight, activity level, gender, and so forth). The clinician may adjust the suggested levels based on other clinical information. In some embodiments, a physician may use the system to create a ramp-up or wean-off plan for a user so that a level of treatment for a user can be modified gradually over time.

In some embodiments, user surveys may be used to determine user adherence to a prescribed plan. User survey results may be used to inform a clinician's course of action at follow up appointments. For example, if the user is not adhering to a treatment plan, the clinician may not adjust the plan at follow up even though physiological indicators are not improving, or a user may indicate that they are struggling with the plan, which may prompt to clinician to adjust the treatment plan so that it is easier to adhere to, for example by eliminating a treatment, switching to a treatment that only needs to be taken once per day, switching to a treatment that imposes fewer dietary restrictions (e.g., a medication that can be taken with or without food), and so forth. In some embodiments, a computer system can be configured to automatically process user survey data and to update a treatment plan based on the user survey data.

In some embodiments, the clinician portal may assign particular users to specific clinicians. This may allow clinicians to develop ongoing relationships with users. In some embodiments, clinicians may be automatically selected based on availability, specialty, or the like. In some embodiments, user health information may be used to determine a clinician to be assigned to the user. For example, a user that has a long history of severe illness may be assigned to a more experienced specialist, or a user with nutritional imbalances may be paired with a qualified nutritionist.

In some embodiments, a physician may use the clinician portal to interact with a user. For example, the clinician portal may include a video communication module, text communication module, and/or audio communication module. In some embodiments, users who use at-home testing may wish to speak with a clinician. For example, a user who has taken a diagnostic test (e.g., a test for strep throat, COVID-19, and so forth), tested for vitamin levels, blood oxygen, allergies, etc., at home during a proctored testing session may be presented with an option to discuss their result with a clinician. If the user chooses to speak with a clinician, then a clinician may receive a notification via the clinician portal and may join an in-progress testing session, for example after the proctor has discussed the results with the user. In some embodiments, the clinician may be able to prescribe treatment remotely based on the results of the at-home test after talking with the user. Similarly, a user may be asked if they wish to speak to a pharmacist if they are prescribed treatment during a testing session, and a pharmacist may use the clinician portal to join the in-progress testing session to discuss treatment. In some embodiments, there may not be a physician or pharmacist available, and the clinician portal may provide a queue of users who need to be called to discuss their test results or treatment. Pharmacists, physicians and other clinicians may then view the queue through the clinician portal and may contact users who have asked to discuss results, treatment plans, and so forth. In some embodiments, the queue can be prioritized based on, for example, urgency of a need for care, severity of symptoms, a time within which a patient needs to receive care (e.g., a user who is traveling soon may be put near the top of the queue), and so forth.

In some embodiments, the clinician portal and/or backend systems may store user health records in an immutable format, for example on a blockchain. In some embodiments, users may be able to authorize a clinician to share subsets of their health data with a specialist, for example if it would be advantageous to consult a specialist for a specific question. In some embodiments, the user may share some or all information with other parties, such as insurance companies, family, employers, other health professionals, and so forth. In some embodiments, a user may choose to make a subset of their information available to a plurality of clinicians, for example if the user would like to receive multiple opinions regarding a particular health issue.

This approach can have many advantages over conventional approaches to sharing medical data. For example, in a conventional scenario, a patient who wanted a second opinion would schedule an appointment with another physician. Once the patient arrives at the physician's office, the patient would be provided with a release form to allow the physician's office to access the patient's records. The physician's office would provide this consent form to the medical facility that has the patient's existing records and would eventually be granted access. If the physician's office and the medical facility use medical record software that is compatible, records can be easily reviewed after access is granted. However, if the medical record software is incompatible or not all records are available in electronic format, records may be difficult to review, may take time to receive, and so forth. For example, the physician's office may receive information in a format that can't be readily and fully integrated into the office's medical record platform, such as PDFs. This can result in significant delays, frustration, and potentially missed information, as physician's struggle to digest information in formats they are not familiar with and the patient must wait for the physician to be granted access to the patient's medical records. Moreover, such an approach can typically result in “all or nothing” access, with the patient having little or no say over exactly what level of access is given to the physician's office. Advantageously, as described herein, patients can grant access to only a subset of medical information. In some embodiments, patients may have full control over which information to grant access to. In some embodiments, patients may have limited control. For example, for proper diagnosis and treatment, it can be important to have a complete and accurate picture of the patient's health as it relates to the condition for a which a second opinion is sought. For example, a physician seeing a patient for a second opinion may need access to both lab results and treatment history related to the condition. If a patient provides lab results but elects not to provide treatment history, the physician would be unable to form an accurate opinion as to the patient's condition, treatment approach, prognosis, and so forth.

In some cases, it may be beneficial for a patient to consult with a specialist. In some embodiments, the specialist may receive medical information and work independently of the clinician. In some embodiments, it can be advantageous for the clinician and specialist to work in a collaborative manner. Similarly, it can be advantageous for a clinician or specialist to share information with a patient in an interactive manner. For example, a provider may show the patient lab results and highlight or circle results that are of particular relevance. Such interactions can help the patient, the specialist, and so forth understand the information. However, such interaction can be difficult in a virtual or remote approach to testing, monitoring, and treatment. Accordingly, there is a need for methods that enable such interactivity and collaboration. Advantageously, collaborative interactions can be implemented in a manner that it suitable for use in low bandwidth settings. This can be especially important for remote healthcare delivery, as remote healthcare can be especially advantageous for those who live in remote or less developed areas where infrastructure and bandwidth may be limited.

Collaborative interaction on webpages can provide several advantages. Collaboration tools, like the ability to draw shapes, command another person's view, etc., are important but often cannot be expected to be part of the website itself. A responsive, low-bandwidth way of adding this functionality can make most web pages collaborative without changing the page's client or server code.

To achieve such collaboration on webpages, a transparent overlay layer can be included. Users can add to and subtract from this layer, show/hide different users' contributions, etc., collaboratively like an online drawing. The overlay can include metadata associated with it. In some embodiments, the overlkay can be sent as an image or series of images to each client and transformed correctly to be displayed based on the size, layout, and scroll location of the client screen. This way, if a user circles part of the webpage, or highlights text, this can be seen by all users regardless of if they are viewing the page on desktop, mobile, or VR/AR headset. Such functionality can be implemented in a variety of ways, for example as a browser extension.

In some embodiments, the overlay can have multiple layers. One layer can be an image that spans the entire webpage, but other layers may be smaller images associated with certain parts of the image, metadata about text the user has highlighted, etc. One advantage to this image and data is that it is very sparse compared to the page itself (mostly empty space). The part that is not empty does not require high resolution, since it may be mostly hand drawn shapes and large text. Therefore, these images can be relatively low resolution with a very high compression ratio. Also, the overlay layer images only need to be updated when a user writes a change to it, meaning it can have a dynamic frame rate that is zero most of the time. This makes maintaining and streaming the overlay as an image much less data and bandwidth intensive than maintaining and streaming the entire web page.

In some embodiments, the overlay can be configured to allow a person to call attention to a specific part of a webpage or the overlay. For example, regardless of client view, the overlay can include a flashing icon associated with the user who is issuing the call to attention. Any other users who click on the icon will have their webpage view shifted in order to see the item of interest (adjust horizontal/vertical scroll automatically, maybe flash the item of interest on arrival).

In some embodiments, the overlay can be configured to allow a person to become the “pilot.” In such cases, any navigation they do can be automatically reflected on all clients who have accepted the other user as the pilot. This could include automatic scrolling to parts of the page, automatic changes to URL or refreshes, etc. In some embodiments, this is not like screen sharing in normal meeting applications. It is more sharing the metadata necessary to command a client's browser to go to the right place. This way the page can remain high resolution and interactive for all users. It will be appreciated that the approaches described herein can be used for any sort of collaboration, whether related to health or not.

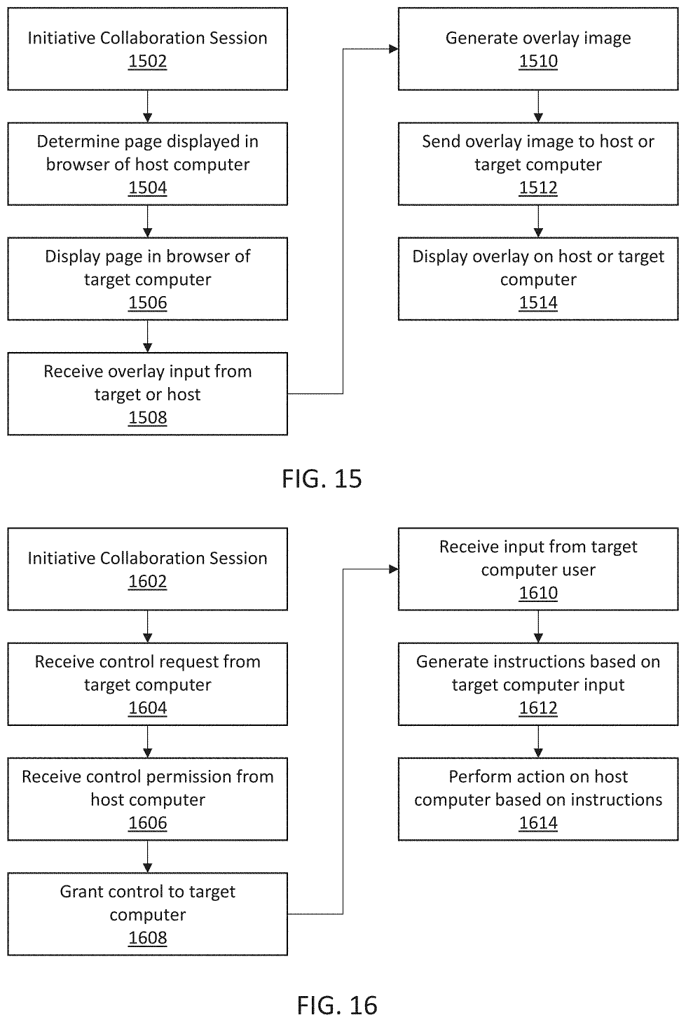

FIG. 15 illustrates an example collaborative process according to some embodiments. As described with reference to FIGS. 15 and 16, a host computer can be a computer that is initially displayed a web page to be shared, and a target computer can be a computer that begins displaying the web page in response to a collaboration session being initiated between the host and target.

At block 1502, a collaboration session can be initiated. In some embodiments, the collaboration session can be initiated by the host computer. In some embodiments, the collaboration session can be initiated by the target computer. At block 1504, the host computer can determine a web page that is displayed in the browser of the host computer. At block 1506, the page can be displayed in the browser of the target computer. In some embodiments, this can happen by, for example, opening up the same web page in the browser of the host computer. In some embodiments, this can be done by showing a image of the web page as it appears in the browser of the target computer. In some embodiments, the target computer can provide the web page contents (e.g., images, HTML, JavaScript, etc.) for rendering and/or execution by the target computer. At block 1508, overlay input can be received from either the target or the host (or both). At block 1510, an image can be generated that indicates the overlay content to be displayed. The computer can also generate information indicating how the image should be displayed (for example, the location on the web page at which the content should be displayed). At block 1512, the overlay image can be transmitted to the host computer or the target computer, depending upon which computer was used to generate the overlay (e.g., if the target created an overlay, it can be sent to the host for display, and vice versa). The transmission can also include instructions for applying the overlay (e.g., a location at which the overlay should be placed). At block 1514, the host or target computer, whichever received the overlay image, can display the overlay in the web page.

While the above description of FIG. 15 discusses the use of overlay images, it will be appreciated that in some embodiments, an overlay may not be in the form of an image. If a user circles something on the screen, writes on the screen, and so forth, then an image may be provided. However, if the user of computer, for example, highlights text, it may be more efficient to generate instructions to highlight the same text on the target computer. For example, the target computer could receive instructions to modify the HTML of the target web page such that the text is highlighted.

FIG. 16 illustrates an example embodiment of a process for sharing control according to some embodiments. The process in FIG. 16 can be useful, for example, to enable a target computer to direct a host computer to scroll a web page, click a button, enter text into a field, select items from a drop down list, alter the state of a checkbox or radio button, or otherwise manipulate content. At block 1602, a collaboration session can be initiated between a target computer and a host computer, for example as described above with reference to FIG. 15. At block 1604, the host computer can receive a control request generated by the target computer. At block 1606, the host computer can grant or deny control permission. If the host computer grants permission, at block 1608 the target computer can be granted permission to control the host (e.g., to manipulate a web page being displayed on the host computer). At block 1610, the target computer can receive input from the user of the target computer. The input can be, for example, touch input, mouse input, keyboard input, and so forth. At block 1612, the target computer can generate instructions based on the target computer input. The instructions can include buttons to be clicked, text to be entered, an amount to be scrolled, and/or other manipulations to perform. At block 1614, the host computer can receive the instructions and, based on the received instructions, perform one or more actions on the host computer.

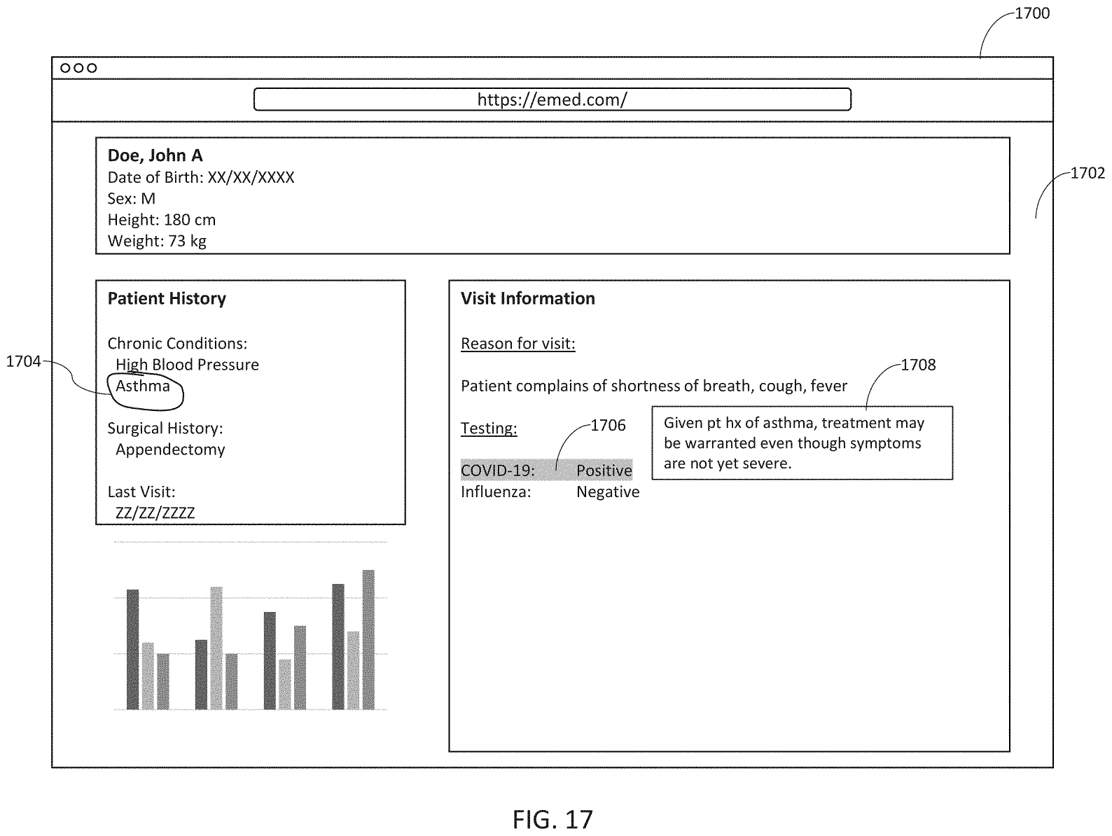

FIG. 17 illustrates an example of overlays according to some embodiments. In FIG. 17, a web browser 1700 displays a web page 1702. The web browser displays two overlays. As illustrated in FIG. 17, the overlay 1704 can be a shape. The shape can be a freeform shape, a rectangle, a square, a circle, an oval, and so forth. For the sake of illustration, it will be assumed that the target computer is creating the overlays, although it will be understood that the host computer could additionally or alternatively be used to produce overlays. The overlay can be sent to the target computer as an image. For example, the image can be an image that includes the underlying content of the web page (in this example, “Asthma”) or could be an imagine that has a transparent background. In some embodiments, the overlay can be provided in a scalar graphics format, such as bitmap, jpeg, portable network graphics, and so forth. In some embodiments, the overlay can be provided in a vector format, such as a scalable vector graphics (svg). The overlay 1706 can indicate that certain text should be highlighted. The overlay 1706 could be provided in the form of an image to replace that part of the web page (e.g., an image that contains the test “COVID-19: Positive” with a colored background. In some embodiments, the overlay 1706 can be provided as a partially transparent image that can be displayed in the overtop of the text on the web page without fully obscuring the text. In some embodiments, the overlay can be provided in the form of HTML or JavaScript that indicates the text should be highlighted. IN some embodiments, an overlay can include a text overlay, such as text overlay 1708. The text overlay 1708 can be provided in the form of an image, HTML, and the like.

It will be appreciated that FIG. 17 is merely exemplary. Other overlays can be included as alternatives or in addition to the overlays depicted in FIG. 17. IN some embodiments, for example, an overlay can be an image. For example, it may be useful to overlay an image showing a chart of a patient's testing results, an image such as an x-ray of the patient's lungs, and so forth.

### Treatment Purchase and Fulfillment

As discussed above, prescription treatment, non-prescription treatment, or both may be appropriate for users of remote or at-home testing, monitoring, and treatment. In some embodiments, for example as described above, a remote health platform can work directly with a pharmacy provider (e.g., pharmaceutical network 150) to fulfill prescriptions. In some embodiments, the pharmacy provider can provide non-prescription treatments, such as over the counter pain relievers, vitamins, supplements, and so forth.

As discussed above, in some cases, a user of a remote or at-home testing platform may receive treatment. It can be important to provide a smooth experience for patients who need to receive treatment, but legal and regulatory concerns can present significant hurdles. According to some embodiments described herein, prescription treatment and fulfillment can be implemented in a manner that reduces the risk of improperly handling protected health information (PHI). The approaches described herein can be used as part of a remote or at-home testing platform. However, the approaches described herein are not limited to remote or at-home testing. For example, according to some embodiments herein, e-commerce websites may implement capabilities for receiving and fulfilling orders for prescription treatment using an online pharmacy network. According to some embodiments, the e-commerce website can avoid the complexities and legal complications associated with collecting PHI, storing prescription drugs, dispensing prescription drugs, prescribing prescription drugs, and so forth, while providing a patient with a seamless shopping experience.

Purchasing prescribed products online on an e-commerce site can be difficult for a variety of reasons. Pharmacies may communicate with insurance companies, healthcare provider platforms, and so forth to receive prescriptions, determine coverage, and so forth. Pharmacies may take measures to ensure that they only dispense products to individuals with valid prescriptions. Pharmacies are also subject to various regulations such as the Health Insurance Portability and Accountability Act (HIPAA) that can impose requirements that are difficult or prohibitively expensive for some sites to meet. For example, compliance may require risk analyses, audits, measures to safeguard protected health information (PHI), employee training, the appointment of a privacy officer, and so forth. Pharmacies that violate these regulations can face large penalties. Thus, many e-commerce sites may be discouraged from offering prescription products. Distribution of prescription products also has various logistical hurdles. Protected health information must be kept secure and only used for permitted purposes, some prescriptions must be temperature controlled, prescription products must be kept secure, and so forth. Given technological, logistical, and regulatory hurdles, many e-commerce sites choose not to offer prescriptions.

An online pharmacy may work by asking a customer to complete a questionnaire that includes information about the individual's identity, symptoms, allergies, medications, and so forth. The responses to the questionnaire may be sent to a pharmacist who can prescribe a product. In some cases, pharmacists may be able to review and approve information in batches. In some embodiments, pharmacists may be able to quickly generate prescriptions, for example with a single click after reviewing the customer's information. The prescription may then be fulfilled from a warehouse that complies with applicable regulations and that is equipped to handle the logistical hurdles of distributing prescription products.

As discussed briefly above, pharmacists can have limited prescribing privileges. Thus, while in some cases, a pharmacist may be able to prescribe a treatment to a patient, in other cases, the pharmacist may interact with another prescriber who has authority to prescribe the treatment.

In some embodiments, an online pharmacy provider may provide a platform that can integrate into third party e-commerce sites. For example, a user may browse a web site, locate a prescription product they are interested in, and add it to their cart. The third-party e-commerce site may then communicate with the online pharmacy provider, who may collect information from the user, issue a prescription, and fulfill the order. The user may submit payment directly to the e-commerce site, rather than to the online pharmacy provider. The online pharmacy provider may collect information using an embedded frame, popup window, application programming interface, and so forth. Advantageously, the third-party e-commerce site does not collect or retain any protected health information and does not ship prescription products, instead relying on the online pharmacy provider. This can have many advantages. For users, a seamless experience may be provided in which a user can purchase a prescription product through a web site that they are familiar with, have an existing account with, etc. For third party e-commerce sites, they can offer a valuable service without incurring the risk of handling protected health information themselves or tackling the obstacles of prescription delivery.

While discussed above in relation to third-party e-commerce websites, the approaches herein can also be used by, for example, a remote or at-home healthcare platform. For example, a remote or at-home healthcare platform may provide testing services but may not otherwise collect health information about a patient, or may isolate data it does collect to reduce the amount of data that can be obtained in the event of a security breach. For example, in some embodiments, the platform may not inquire or store data about, for example, other conditions that the patient has, medications that the patient is taking, and so forth. According to some embodiments, the platform can reduce or eliminate the storage of protected health information by instead working with a pharmacy network that can directly receive the protected health information from the patient. In some embodiments, the approaches herein can be deployed to other types of web sites, intranet sites, mobile applications, and so forth. For example, an employee intranet page may include functionality that enables an employee who has a positive test result (e.g., for influenza or COVID-19) to order treatment.

Approaches similar to those described herein can also be used for other purposes, such as sharing information with a medical professional (e.g., provider network 140). For example, a patient may access a remote or at-home testing platform (e.g., through an app or web page). In some embodiments, the patient's PHI (or a subset thereof) can be shared with a provider. In some embodiments, the patient's PHI (or a subset thereof) may not be shared with the provider. Instead, the remote or at-home testing platform may provide an interface to the patient that appears seamless to the patient but which directs PHI directly to the provider without the information being stored by the remote or at-home testing platform.

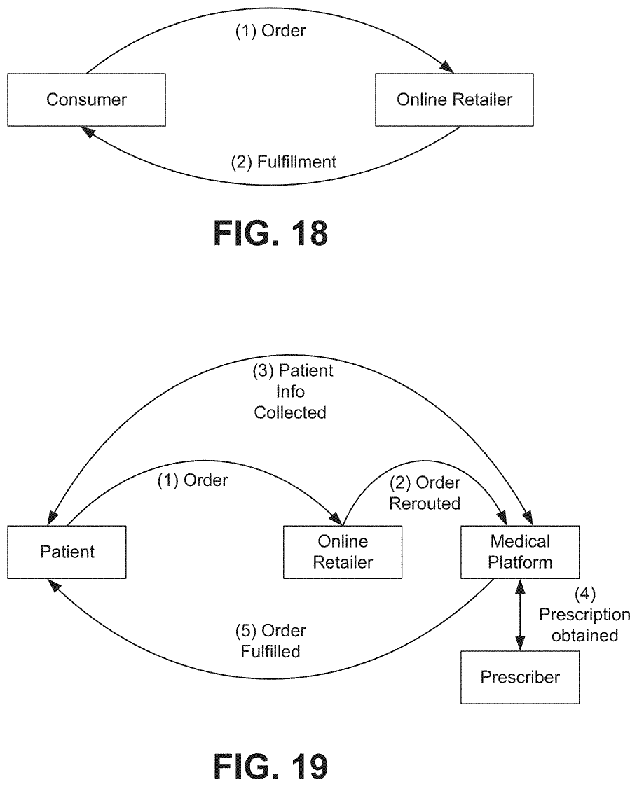

FIG. 18 depicts a normal online retail scenario. At (1), a consumer places an order with an online retailer, and, at (2), the online retailer ships the order to the consumer. In this scenario, the online retailer does not offer prescription products and has no mechanism for providing a prescription.

FIG. 19 depicts a system according to some embodiments in which an online retailer can offer prescription products. At (1), a patient places an order with an online retailer. At (2), the online retailer reroutes the order (or part of the order containing a prescription product) to a medical platform (e.g., an online pharmacy platform) for fulfillment of the prescription order. At (3), the medical platform collects patient information from the patient for obtaining a prescription and fulfilling the order. The medical platform, at (4), contacts a prescriber who provides a prescription. The provider may be, for example, a pharmacist, nurse practitioner, medical doctor, and so forth. In some embodiments, the medical platform may be configured to select a prescriber based at least in part on the prescription product and the qualifications and/or certifications of the providers. For example, one prescription product may be prescribed by a pharmacist, while another prescription product may require a doctor or nurse practitioner to issue the prescription. The medical platform, at (5), fulfills the order and sends the prescription product to the patient directly.

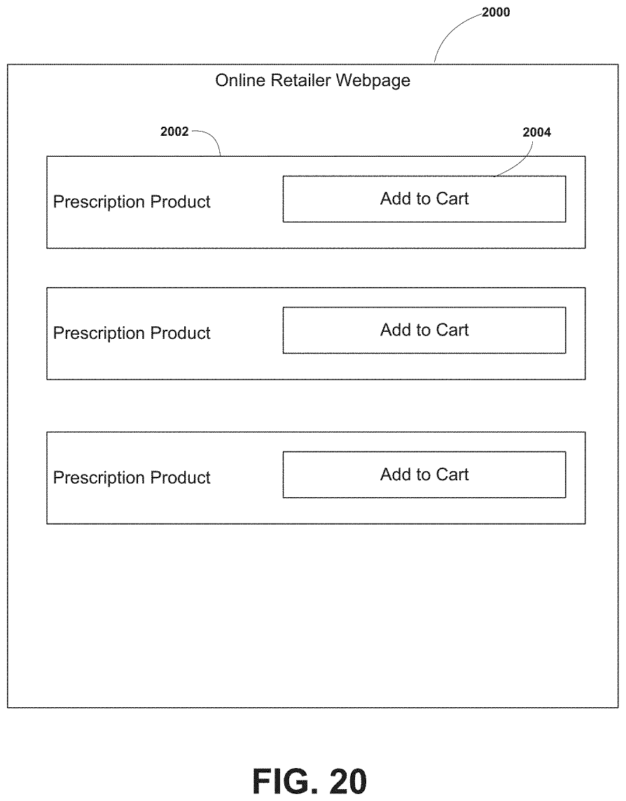

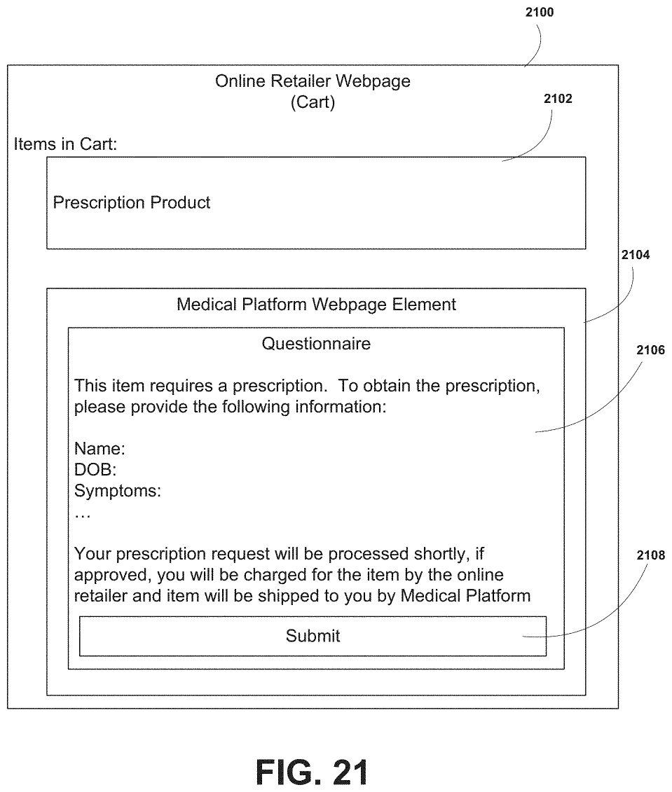

FIG. 20 depicts an example web page 2000 that may be provided by a third-party e-commerce site. As depicted in FIG. 17, the site may offer a variety of prescription products 2002, and a user of the site may be able to add prescription products to their cart by simply clicking the “Add to Cart” button 2004. Thus, to the user, ordering a prescription product may operate largely the same as ordering any other product from the e-commerce site. This may provide convenience for the user who can order prescriptions when ordering other products, and who can pay for prescriptions using the e-commerce site.

It will be appreciated that such an interface is not limited to third-party e-commerce sites. For example, a remote or at-home testing platform can provide a similar page to users who receive a positive test result. For example, a user who tests positive for COVID-19 may be presented with a page that allows them to add prescription treatments to a cart. In some embodiments, the medical platform may only provide prescription products. In some embodiments, the medical platform may provide both prescription and non-prescription products. While not all e-commerce sites may be interested in such functionality (for example, such functionality may cut into the e-commerce sites' direct sales, which could impact profits), other entities may be interested in offering such functionality. For example, a remote or at-home testing platform provider may wish to give patients the option of buying over the counter treatments at the same time the patient is ordering a prescription medication (e.g., as part of the same shopping transaction).

FIG. 18 shows an example web page that may be presented to a user of a third-party e-commerce site according to some embodiments. The user may visit a web page 2100, such as the shopping cart. The medical platform webpage element 2100 can include a list 2102 of items in the user's cart. In some embodiments, the cart can include only prescription items. In some embodiments, the cart can include a mix of prescription and non-prescription items. In some embodiments, the items can all be provided by a medical platform. In some embodiments, some of the items (e.g., non-prescription items) can be provided by the e-commerce site. In some embodiments, a medical platform webpage element 2104 may be embedded in the medical platform webpage element 2100. In some embodiments, the user may be automatically directed to a page containing the medical platform webpage element 2100 when adding a prescription product to their cart. The medical platform webpage element 2100 may present a questionnaire 2106 to collect information needed to obtain the prescription, such as the user's name, date of birth, symptoms, insurance coverage, and so forth. The medical platform webpage element 2104 can include a button 2108 to submit the questionnaire. The medical platform webpage element may explain to the user that a prescription is needed and that someone will review their information, that the e-commerce site will handle billing, and so forth.

### Computer Systems

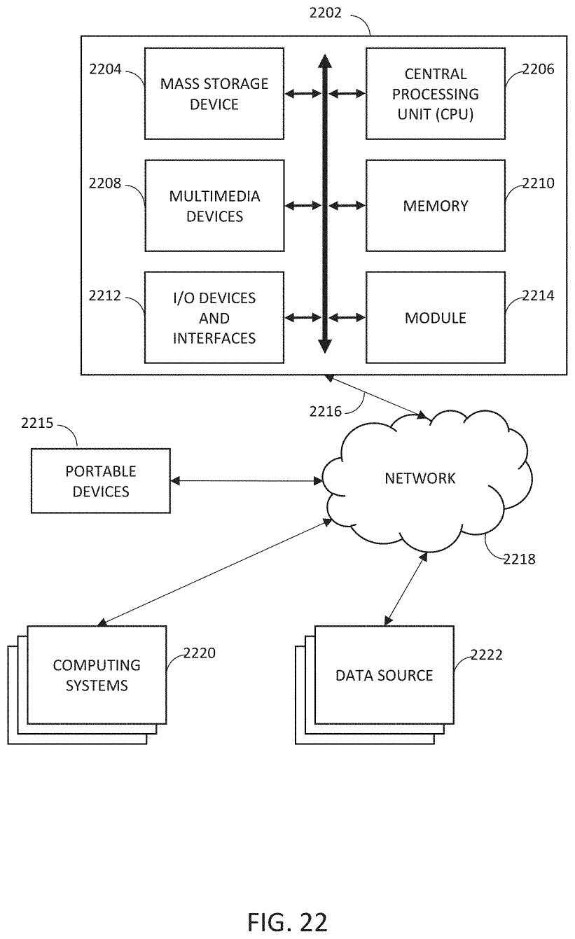

FIG. 22 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 22. The example computer system 2202 is in communication with one or more computing systems 2220 and/or one or more data sources 2222 via one or more networks 2218. While FIG. 22 illustrates an embodiment of a computing system 2202, it is recognized that the functionality provided for in the components and modules of computer system 2202 may be combined into fewer components and modules, or further separated into additional components and modules.

The computer system 2202 can comprise a module 2214 that carries out the functions, methods, acts, and/or processes described herein. The module 2214 is executed on the computer system 2202 by a central processing unit 2206 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 2202 includes one or more processing units (CPU) 2206, which may comprise a microprocessor. The computer system 2202 further includes a physical memory 2210, such as random-access memory (RAM) for temporary storage of information, a read only memory (ROM) for permanent storage of information, and a mass storage device 2204, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 2202 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 2202 includes one or more input/output (I/O) devices and interfaces 2212, such as a keyboard, mouse, touch pad, and printer. The I/O devices and interfaces 2212 can include one or more display devices, such as a monitor, that allows the visual presentation of data to a user. More particularly, a display device provides for the presentation of GUIs as application software data, and multi-media presentations, for example. The I/O devices and interfaces 2212 can also provide a communications interface to various external devices. The computer system 2202 may comprise one or more multi-media devices 2208, such as speakers, video cards, graphics accelerators, and microphones, for example.

The computer system 2202 may run on a variety of computing devices, such as a server, a Windows server, a Structure Query Language server, a Unix Server, a personal computer, a laptop computer, and so forth. In other embodiments, the computer system 2202 may run on a cluster computer system, a mainframe computer system and/or other computing system suitable for controlling and/or communicating with large databases, performing high volume transaction processing, and generating reports from large databases. The computing system 2202 is generally controlled and coordinated by an operating system software, such as Windows XP, Windows Vista, Windows 22, Windows 8, Windows 220, Windows 221, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 2202 illustrated in FIG. 22 is coupled to a network 2218, such as a LAN, WAN, or the Internet via a communication link 2216 (wired, wireless, or a combination thereof). Network 2218 communicates with various computing devices and/or other electronic devices. Network 2218 is communicating with one or more computing systems 2220, one or more portable devices 2215, and/or one or more data sources 2222. The module 2214 may access or may be accessed by computing systems 2220, portable devices 2215 and/or data sources 2222 through a web-enabled user access point. Connections may be a direct physical connection, a virtual connection, and other connection type. The web-enabled user access point may comprise a browser module that uses text, graphics, audio, video, and other media to present data and to allow interaction with data via the network 2218.

Access to the module 2214 of the computer system 2202 by computing systems 2220, portable devices 2215, and/or by data sources 2222 may be through a web-enabled user access point such as the computing systems' 2220, portable devices' 2215, or data source's 2222 personal computer, cellular phone, smartphone, laptop, tablet computer, e-reader device, audio player, or another device capable of connecting to the network 2218. Such a device may have a browser module that is implemented as a module that uses text, graphics, audio, video, and other media to present data and to allow interaction with data via the network 2218.

The output module may be implemented as a combination of an all-points addressable display such as a cathode ray tube (CRT), a liquid crystal display (LCD), a plasma display, or other types and/or combinations of displays. The output module may be implemented to communicate with input devices 2212 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 2202 may comprise a physical or logical connection established between a remote microprocessor and a mainframe host computer for the express purpose of uploading, downloading, or viewing interactive data and databases on-line in real time. The remote microprocessor may be operated by an entity operating the computer system 2202, including the client server systems or the main server system, an/or may be operated by one or more of the data sources 2222, one or more of the computing systems 2220, and/or one or more of the portable devices 2215. In some embodiments, terminal emulation software may be used on the microprocessor for participating in the micro-mainframe link.

In some embodiments, computing systems 2220 who are internal to an entity operating the computer system 2202 may access the module 2214 internally as an application or process run by the CPU 2206.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the user. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 2202 may include one or more internal and/or external data sources (for example, data sources 2222). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Cache), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 2202 may also access one or more databases 2222. The databases 2222 may be stored in a database or data repository. The computer system 2202 may access the one or more databases 2222 through a network 2218 or may directly access the database or data repository through I/O devices and interfaces 2212. The data repository storing the one or more databases 2222 may reside within the computer system 2202.

FIG. 23 is a block diagram illustrating an example embodiment of a computer system configured to run software for implementing one or more embodiments of the health testing and diagnostic systems, methods, and devices disclosed herein. In some embodiments, the various systems, methods, and devices described herein may also be implemented in decentralized systems such as, for example, blockchain applications. For example, blockchain technology may be used to maintain user profiles, insurance profiles, test results, test site databases, and/or financing databases or ledgers, dynamically generate, execute, and record testing plan agreements, perform searches, conduct patient-health professional matching, determine pricing, and conduct any other functionalities described herein.

In some embodiments, a health testing, diagnostic, and treatment platform 2302 may be comprised of a registration and purchase module 2304, a testing module 2306, an analytics module 2308, and a reporting module 2310. The health testing, diagnostic, and treatment platform 2302 may also comprise a consumer profile database 2312, a test database 2314, and/or a site database 2316. The health testing, diagnostic, and treatment platform 2302 can be connected to a network 2320. The network 2320 can be configured to connect the health testing, diagnostic, and treatment platform 2302 to one or more delivery service systems 2322, one or more forecast data supplier systems 2324, one or more insurance provider devices 2326, one or more doctor network systems 2328, one or more pharmaceutical network systems 2330, one or more test manufacturer systems 2332, and/or one or more consumer devices 2334.

The registration and purchase module 2304 may function by facilitating consumer registration through one or more registration interfaces and in conjunction with the consumer profile database 2312, store consumer registration data. The testing module 2306 may be configured to allow a consumer to initiate and complete a medical test, as described herein. The analytics module 2308 may be configured to dynamically analyze patient tests across a given population stored in the test database 2314 and provide structured data of the test results. The reporting module 2310 may function by dynamically and automatically reporting test results insurance provider systems, and/or third parties using one or more interfaces, such as one or more application programming interfaces. Each of the modules can be configured to interact with each other and the databases discussed herein.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein. Accordingly, the claims are not intended to be limited to the embodiments shown herein but are to be accorded the widest scope consistent with this disclosure, the principles and the novel features disclosed herein.

What is claimed is:

1. A computer system for at-home diagnostic testing and treatment, the computing system comprising:
one or more computer data stores configured to store a plurality of computer executable instructions; and
one or more hardware computer processors in communication with the one or more computer data stores and configured to execute the plurality of computer executable instructions in order to cause the computer system to:
receive, from a first data source, forecast information, wherein the forecast information indicates a projected infection trend;
receive, from a second data source, a risk profile;
receive, from a third data source, a selection of one or more individuals;
convert the selection of one or more individuals to a format compatible with a fourth data source;
request, from the fourth data source, health information associated with at least one of the one or more individuals;
receive, from the fourth data source, health information associated with at least one of the one or more individuals;
determine, for each of the one or more individuals, a risk score, wherein the risk score is based at least in part on the health information and the forecast information; and
determine that the risk score of an individual of the one or more individuals meets the risk profile.

2. The computer system of claim 1, wherein the computer executable instructions are further configured to cause the computer system to:
electronically communicate a request to send a test box to the individual, wherein the request is based on the risk score exceeding a risk threshold.

3. The computer system of claim 1, wherein receiving the selection of one or more individuals comprises:
providing a map display, wherein the map display is configured to show a visual representation of the forecast information;
receiving a selection of a geographic area; and
determining one or more individuals in the geographic area.

4. The computer system of claim 1, wherein receiving health information for one or more individuals comprises:
determining that a query return multiple results for an individual of the one or more individuals; and
determining which of the multiple results matches the individual.

5. The computer system of claim 1, wherein receiving health information for one or more individuals comprises:
determining a confidence level for health information associated with each of the one or more individuals.

6. The computer system of claim 1, wherein the computer data store further comprises instructions that, when executed by the one or more computer hardware processors, causing the computer system to:
convert the selection of one or more individuals to a format compatible with a fifth data source;
request, from the fifth data source, non-health information associated with at least one of the one or more individuals; and
receive, from the fifth data source, non-health information associated with at least one of the one or more individuals,
wherein the risk score is additionally determined based at least in part on the non-health information.

7. The computer system of claim 6, wherein the non-health information comprises one or more of public transit information, pharmacy location information, employment information, housing information, healthcare provider location information, or income information.

8. The computer system of claim 1, wherein determining that the risk score of an individual of the one or more individuals meets the risk profile comprises determining that the risk score is above a threshold amount.

9. The computer system of claim 1, wherein the risk score indicates a likelihood that the individual will suffer a severe health consequence if the individual contracts an infectious disease.

10. The computer system of claim 1, wherein determining the risk score comprises:
providing the health information and the forecast information to a machine learning model; and
receiving output from the machine learning model, the output indicating a risk level for an infectious disease.

11. A method for at-home diagnostic testing and treatment, the computing system comprising:
receiving, from a first data source, forecast information, wherein the forecast information indicates a projected infection trend;
receiving, from a second data source, a risk profile;
receiving, from a third data source, a selection of one or more individuals;
converting the selection of one or more individuals to a format compatible with a fourth data source;
requesting, from the fourth data source, health information associated with at least one of the one or more individuals;
receiving, from the fourth data source, health information associated with at least one of the one or more individuals;
determining, for each of the one or more individuals, a risk score, wherein the risk score is based at least in part on the health information and the forecast information; and
determining that the risk score of an individual of the one or more individuals meets the risk profile.

12. The method of claim 11, further comprising:
electronically communicate a request to send a test box to the individual.

13. The method of claim 11, wherein receiving the selection of one or more individuals comprises:
providing a map display, wherein the map display is configured to show a visual representation of the forecast information;
receiving a selection of a geographic area; and
determining one or more individuals in the geographic area.

14. The method of claim 11, wherein receiving health information for one or more individuals comprises:
determining that a query return multiple results for an individual of the one or more individuals; and
determining which of the multiple results matches the individual.

15. The method of claim 11, wherein receiving health information for one or more individuals comprises:
determining a confidence level for health information associated with each of the one or more individuals.

16. The method of claim 11, further comprising:
converting the selection of one or more individuals to a format compatible with a fifth data source;
requesting, from the fifth data source, non-health information associated with at least one of the one or more individuals; and
receiving, from the fifth data source, non-health information associated with at least one of the one or more individuals,
wherein the risk score is additionally determined based at least in part on the non-health information.

17. The method of claim 16, wherein the non-health information comprises one or more of public transit information, pharmacy location information, employment information, housing information, healthcare provider location information, or income information.

18. The method of claim 11, wherein determining that the risk score of an individual of the one or more individuals meets the risk profile comprises determining that the risk score is above a threshold amount.

19. The method of claim 11, wherein the risk score indicates a likelihood that the individual will suffer a severe health consequence if the individual contracts an infectious disease.

20. The method of claim 11, wherein determining the risk score comprises:
providing the health information and the forecast information to a machine learning model; and
receiving output from the machine learning model, the output indicating a risk level for an infectious disease.
